(19) United States
(12) Patent Application Publication
(10) Pub. No.: US 2026/0151186 A1
(43) Pub. Date: Jun. 4, 2026

(54) SYSTEMS AND METHODS FOR CLASSIFICATION OF TOTAL JOINT ARTHROPLASTY PATIENT ALIGNMENT BASED ON INTRAOPERATIVE DATA ACQUISITIONS

(71) Applicant: Advita Ortho, LLC, Gainesville, FL (US)

(72) Inventors: Laurent Angibaud, Gainesville, FL (US); Prudhvi Chinimilli, Milpitas, CA (US)

(21) Appl. No.: 19/384,610

(22) Filed: Nov. 10, 2025

Related U.S. Application Data
(60) Provisional application No. 63/718,827, filed on Nov. 11, 2024.

(51) Int. Cl. A61B 34/10; A61B 5/00; A61B 34/00; A61B 34/20

(52) U.S. Cl. CPC A61B 34/10; A61B 5/7264; A61B 34/20; A61B 34/25; A61B 2034/102; A61B 2034/2046

(57) ABSTRACT

A system and method for joint arthroplasty includes capturing patient-specific three-dimensional joint data during a first arthroplasty stage and utilizing a joint phenotype classification algorithm to predict a likelihood of a phenotype match or transition between first and second arthroplasty stages. The algorithm selects at least one joint-specific metric from the pre-cut data, transforms the metric into a feature vector in an N-dimensional feature space, assigns the feature vector to at least one patient-specific pre-cut cluster trained on data from other patients, determines a pre-cut joint phenotype, predicts at least one patient-specific post-cut cluster and a post-cut joint phenotype. The system compares pre-cut and post-cut phenotypes to compute a likelihood of a match or transition, and outputs the likelihood on a user interface intraoperatively to facilitate verification that a planned bone cut will achieve a desired clinical target, or modification of implant parameters, surgical parameters, or soft-tissue balance, for continuing the procedure.

### FIELD OF TECHNOLOGY

The present disclosure generally relates to orthopedic surgery, and more particular to systems and methods for classification of total joint arthroplasty patient alignment based on intraoperative data acquisitions.

### BACKGROUND OF TECHNOLOGY

Total knee arthroplasty (TKA) is widely recognized as a safe and effective treatment for end-stage knee arthritis. It is regarded as one of the most successful procedures in modern medicine, with over 90% of patients experiencing significant pain relief and improved ability to perform daily activities following surgery. Despite these positive outcomes, up to 20% of patients remain dissatisfied with their TKA results.

The high percentage of dissatisfaction among TKA patients may be attributed to the use of systematic alignment techniques, such as neutral alignment, applied uniformly to each patient. This one-size-fits-all approach may be overly simplistic and fails to adequately account for individual anatomical and functional variations. At the same time, the optimal alignment defining a personalized target for each TKA remains uncertain.

Thus, there is a need in the art for a more personalized TKA alignment approach and a better understanding of the alignment variability in native knees utilizing a more advanced system to classify the alignment seems necessary.

### SUMMARY

In at least some embodiments, a method may include initiating a joint arthroplasty procedure for implantation of an implant into a joint of a patient based on at least one planned bone cut of at least one bone member of the joint; may include capturing patient-specific three-dimensional (3D) data of the joint of the patient during a first arthroplasty stage of the joint arthroplasty procedure; may include utilizing at least one joint phenotype classification algorithm to predict, based on the patient-specific 3D data of the joint of the patient captured during the first arthroplasty stage, a likelihood of a joint phenotype match or a joint phenotype transition between a patient-specific joint phenotype in the first arthroplasty stage and a patient-specific joint phenotype in a second arthroplasty stage of the joint arthroplasty procedure; where the at least one joint phenotype classification algorithm may be configured to: may select, from the patient-specific 3D data, at least one joint-specific metric for the first arthroplasty stage; may transform each of the at least one joint-specific metric into at least one feature vector in an N-dimensional feature space; may assign the at least one feature vector to at least one patient-specific pre-cut cluster of a plurality of pre-cut clusters generated during a training of the at least one joint phenotype classification algorithm based on 3D data of a plurality of other patients; may determine the patient-specific joint phenotype for the first arthroplasty stage based on the at least one patient-specific pre-cut cluster; may predict for the patient, based on the at least one patient-specific pre-cut cluster, at least one patient-specific post-cut cluster based at least in part on the plurality of other patients; may determine the patient-specific joint phenotype for the second arthroplasty stage based on the at least one patient-specific post-cut cluster; and may compare the patient-specific joint phenotype at the first arthroplasty stage with the patient-specific joint phenotype at the second arthroplasty stage to determine the likelihood of the joint phenotype match or the joint phenotype transition therebetween; and may include outputting on a graphical user interface during the joint arthroplasty procedure, the likelihood of the joint phenotype match or the joint phenotype transition to facilitate: may verify in the first arthroplasty stage that the at least one planned bone cut of the at least one bone member of the joint will achieve a desired clinical target, or may modify in the first arthroplasty stage at least one of: at least one implant parameter of the implant in the joint, at least one surgical parameter of the at least one planned bone cut, or an adjustment of a soft tissue balance of the joint; and may include continuing the joint arthroplasty procedure from the first arthroplasty stage to the second arthroplasty stage by performing the at least one planned bone cut of the at least one bone member of the joint based on the modification or the verification.

In at least some embodiments, a method may include initiating a joint arthroplasty procedure for implantation of an implant into a joint of a patient based on at least one planned bone cut of at least one bone member of the joint; may include capturing patient-specific three-dimensional (3D) data of the joint of the patient during a first arthroplasty stage of the joint arthroplasty procedure; may include utilizing at least one joint phenotype classification algorithm to predict, based on the patient-specific 3D data of the joint of the patient captured during the first arthroplasty stage, a likelihood of a joint phenotype match or a joint phenotype transition between a patient-specific joint phenotype in the first arthroplasty stage and a patient-specific joint phenotype in a second arthroplasty stage of the joint arthroplasty procedure; where the at least one joint phenotype classification algorithm may be configured to select, from the patient-specific 3D data, at least one joint-specific metric for the first arthroplasty stage; may transform each of the at least one joint-specific metric into at least one feature vector in an N-dimensional feature space; may assign the at least one feature vector to at least one patient-specific pre-cut cluster of a plurality of pre-cut clusters generated during a training of the at least one joint phenotype classification algorithm based on 3D data of a plurality of other patients; may determine the patient-specific joint phenotype for the first arthroplasty stage based on the at least one patient-specific pre-cut cluster; may predict for the patient, based on the at least one patient-specific pre-cut cluster, at least one patient-specific post-cut cluster based at least in part on the plurality of other patients; may determine the patient-specific joint phenotype for the second arthroplasty stage based on the at least one patient-specific post-cut cluster; and may compare the patient-specific joint phenotype at the first arthroplasty stage with the patient-specific joint phenotype at the second arthroplasty stage to determine the likelihood of the joint phenotype match or the joint phenotype transition therebetween; and may include outputting on a graphical user interface during the joint arthroplasty procedure, the likelihood of the joint phenotype match or the joint phenotype transition to facilitate a verification in the first arthroplasty stage that the at least one planned bone cut of the at least one bone member of the joint will achieve a desired clinical target, or a modification in the first arthroplasty stage of at least one of at least one implant parameter of the implant in the joint, at least one surgical parameter of the at least one planned bone cut, or an adjustment of a soft tissue balance of the joint; and may include continuing the joint arthroplasty procedure according to the modification or the verification based on the likelihood of the phenotype match or the phenotype transition.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 is a platform combining a computer assisted orthopedic system (CAOS) and an intra-articular tensioner device allowing the acquisition of the compartment-specific knee joint gaps throughout the full range of motion under force-controlled distraction maintained by two antagonist mechanical actuators in accordance with one or more embodiments of the present disclosure;

FIGS. 2A-2J are diagrams illustrating stages in a tibia-first surgical workflow in accordance with one or more embodiments of the present disclosure;

FIG. 3 are graphs illustrating a transformation of a 2D dynamic trajectory (VV and flexion) into a single point in a three-dimensional feature space in accordance with one or more embodiments of the present disclosure;

FIGS. 4A-4C are graphs illustrating clustering results for pre-cut and post-cut data using k-means with an optimal 8-dimensional feature space based on VV angles at specific flexion points in accordance with one or more embodiments of the present disclosure;

FIGS. 5A-5B are heat maps displaying the distribution of clusters and evolution classes for 1087 cases under pre-cut and post-cut conditions in accordance with one or more embodiments of the present disclosure;

FIGS. 6A-6D are diagrams illustrating a 2D visualization of pre-cut kinematics conditions in accordance with one or more embodiments of the present disclosure;

FIGS. 7A-7B are diagrams illustrating a tree visualization of the 11 dominant dynamic knee phenotypes, showing cluster distributions at the top level and evolution distributions at the lower level for pre-cut and post-cut data in accordance with one or more embodiments of the present disclosure;

FIGS. 8A-8B are Sankey diagrams illustrating the transitions from pre-cut to post-cut dynamic knee phenotypes across 1087 cases in accordance with one or more embodiments of the present disclosure;

FIG. 9 are tables presenting pre-cut to post-cut dynamic knee phenotype transitions in tabular form, with cluster-level transitions and evolution-level transitions in accordance with one or more embodiments of the present disclosure;

FIGS. 10A-10B illustrates transitions for a high-volume Surgeon A (113 cases) with Sankey diagrams and transition tables in accordance with one or more embodiments of the present disclosure;

FIGS. 11A-11B illustrates transitions for a low-volume Surgeon B (18 cases) with Sankey diagrams and transition tables in accordance with one or more embodiments of the present disclosure;

FIG. 12 is a table displaying further details of a distribution of evolution classes for both pre-cut and post-cut conditions;

FIG. 13 depicts a block diagram of an exemplary computer-based system/platform in accordance with one or more embodiments of the present disclosure;

FIG. 14 depicts a block diagram of another exemplary computer-based system/platform in accordance with one or more embodiments of the present disclosure;

FIGS. 15 and 16 are diagrams illustrating implementations of cloud computing architecture/aspects with respect to which the disclosed technology may be specifically configured to operate, in accordance with one or more embodiments of the present disclosure;

FIG. 17 schematically illustrates an operating room using an improved computer-based surgery assistance device for classification of total joint arthroplasty patient alignment based on intraoperative data acquisitions in accordance with one or more embodiments of the present disclosure;

FIG. 18 is a block diagram of the controller of an improved computer-based platform for classification of total joint arthroplasty patient alignment based on intraoperative data acquisitions in accordance with one or more embodiments of the present disclosure; and

FIG. 19 is a flowchart of a method for classification of total joint arthroplasty patient alignment based on intraoperative data acquisitions in accordance with one or more embodiments of the present disclosure.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “In at least some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In at least some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

With regard to lower-limb alignment classification approaches, the coronal alignment of the knee may be classified into three classes: neutral, varus, and valgus. This traditional approach, however, may be overly simplistic and may not account for individual morphological variations. Growing interest in individualized alignment techniques such as kinematic alignment (KA), restricted KA, inverse KA, and functional alignment (FA) may lead to several radiological classification systems that aim to categorize coronal alignment of knee phenotypes during weight bearing.

A common feature among these classification methods may be the use of double-leg, weight-bearing, and/or long-leg radiographs, representing a static modality. These measurements taken preoperatively may guide surgeons in making intraoperative decisions. The major limitation of these methods is that they do not consider dynamic variations in the knee alignment, such as changes in hip-knee-ankle (HKA) angle that may occur as the knee moves from extension to flexion for activities like walking (gait cycle). Thus, the assumption that standing coronal alignment remains constant throughout the range of motion may be controversial. A recent study demonstrated that the dynamic HKA (dHKA) angle measured throughout the gait cycle showed low to moderate correlation with static HKA angle in varus knees and no correlation in valgus knees. This finding underscores the need for systems and methods to classify knee alignment based on dynamic measurements across the full range of motion, rather than relying solely on static images.

Preoperative systems may include laboratory-based systems such as an optimal motion capture system, that enable the tracking of 3D joint kinematics during various activities. These systems, considered the gold standard in kinematic measurement, may involve placing trackers on the body and capturing movement using infrared cameras. While they provide highly accurate and comprehensive data on joint movement, they are expensive, time consuming to set up, and require a controlled environment, which may be cumbersome for patients. In addition, because of the non-invasive aspect of these technologies, these acquisitions may not correlate with those acquired during the surgery using the probing of the bone members.

In contrast, wearable devices may offer a more practical, low-cost alternative for capturing joint kinematics. These compact sensors may be easily worn outside the lab, providing more flexibility in data collection. However, they lack accuracy and may be highly dependent on precise sensor placement.

Intraoperative systems, such as computer assisted orthopedic systems (CAOS), may provide real-time guidance during surgery, thus enabling surgeons to make precise adjustments based on patient's unique anatomy and joint dynamics. Unlike preoperative systems, CAOS may offer real-time feedback, which may be used to both perform acquisitions, develop a surgical plan based on these acquisitions, and then to guide the execution of the defined surgical plan. This capability may allow surgeons to directly monitor and make decisions on alignment, balance, and/or laxity throughout the motion.

As detailed herein at least one technical problem being addressed is that discrepancies may arise when comparing measurements such as HKA angle obtained through CAOS with those from preoperative system such as motion capture lab and/or wearables. Differences in technology and data acquisition methods between preoperative and intraoperative systems may contribute to these inconsistencies. Consequently, relying solely on preoperative assessment for making intraoperative decisions may not be optimal.

In at least some embodiments, at least one technical solution described herein may involve providing real-time, intraoperative systems that may help to minimize these discrepancies, allowing more precise surgical planning. The need for an intraoperative classification system may be based on dynamic acquisition whereby the limitations of current static preoperative classification systems may underscore the need for an intraoperative classification system that may incorporate dynamic measurements, such as dHKA (i.e., 3D HKA) angle acquired throughout the knee's full range of motion. This system may provide a more accurate assessment of the knee alignment by capturing real-time kinematic data that reflects how the knee functions during actual movement, rather than relying on a single static snapshot. By classifying TKA patients based on dynamic acquisitions, surgeons may have access to a comprehensive view of each patient's unique alignment patterns, allowing them to personalize the procedure more precisely.

At least some embodiments of the present disclosure herein describe an advanced classification system for total joint arthroplasty patient alignment, that may leverage dynamic acquisitions captured by the CAOS technology intraoperatively. This classification system aims to address several important aspects that would improve surgical accuracy, enhance personalized alignment techniques, and offer valuable insights into surgical practices.

In at least some embodiments, the system for classification of total joint arthroplasty patient alignment may process dynamic acquisitions captured by CAOS including three-dimensional alignment data (e.g., 3D HKA angles when applied to TKA) and other relevant joint metrics intraoperatively. The system may implement classification algorithms that may categorize dynamic joint motion patterns into clinically relevant phenotypes across different joint types. By analyzing full dynamic joint motion ranges, these classification algorithms may allow for a comprehensive classification of alignment and movement behaviors, that may support personalized alignment strategies tailored to each patient's unique biomechanics.

In at least some embodiments, the system may provide real-time verification of dynamic joint phenotypes (i.e., 3D phenotypes) whereby dynamic acquisitions may be typically performed at multiple stages intraoperatively throughout surgeries both before (pre-cuts) and after (post-cuts) bone cuts. During these acquisitions, the joint may be manipulated through its full range of motion, and the CAOS may capture three-dimensional kinematics data. For personalized alignment, surgeons may aim to replicate each patient's native alignment, thus making it essential to match post-cut (or post-implant) alignment to pre-cut alignment. In some extreme case of deformity, it may be advantageous to intentionally correct the post-cut (or post-implant) alignment compared to the pre-cut alignment. However, directly interpreting dynamic trajectories intraoperatively may be challenging for surgeons due to data complexity. The classification algorithm may provide real-time feedback on whether post-cut dynamic alignment matches with pre-cut dynamic alignments in terms of dynamic joint phenotypes.

In at least some embodiments, the system may further provide insight into an evolution of surgical alignment philosophies. The classification algorithms may also be applied to historical case data, thus enabling retrospective analyses that may reveal trends and variations in surgical alignment practices. For example, the system may assess how often post-cut dynamic alignments may be historically matched with pre-cut dynamic alignments. This may provide objective insights into evolving surgical philosophies and alignment preferences, potentially informing future surgical planning, training, and refinement of alignment techniques.

In at least some embodiments, the system may provide guidance on dynamic joint phenotypes matching for junior surgeons. The classification system may be a valuable training and guidance tool for junior surgeons, allowing them to gain insight into the alignment practices of experienced surgeons. By analyzing dynamic joint phenotypes data from the expert-led cases, the system may be configured to highlight alignment patterns that experienced surgeons achieve in second arthroplasty stages. This data in the form of dynamic joint phenotypes may assist junior surgeons to understand the best practices to perform dynamic alignment matching.

In at least some embodiments, a novel method to improve clinical outcomes may be based on the following process that may be implemented by at least one processor. The process may include receiving pre-operative, intra-operative, and/or post-operative data from a group of patients. For each patient, the classification algorithm may be applied to characterize each patient in at least at one first arthroplasty stage. For each patient, the classification algorithm may be applied to characterize each patient in at least at one second arthroplasty stage. For each patient, a classification change or match from the pre-cut characterization to the post-cut characterization may be determined. For each patient, the evolution of a clinical score may be compared or monitored from the pre-operative stage to the post-operative stage. The data may be processed to establish optimal guidance as to how treat a patient based on his/her pre-cut characterization, so it may translate into the definition of a surgical plan intended to achieve optimal post-cut characterization.

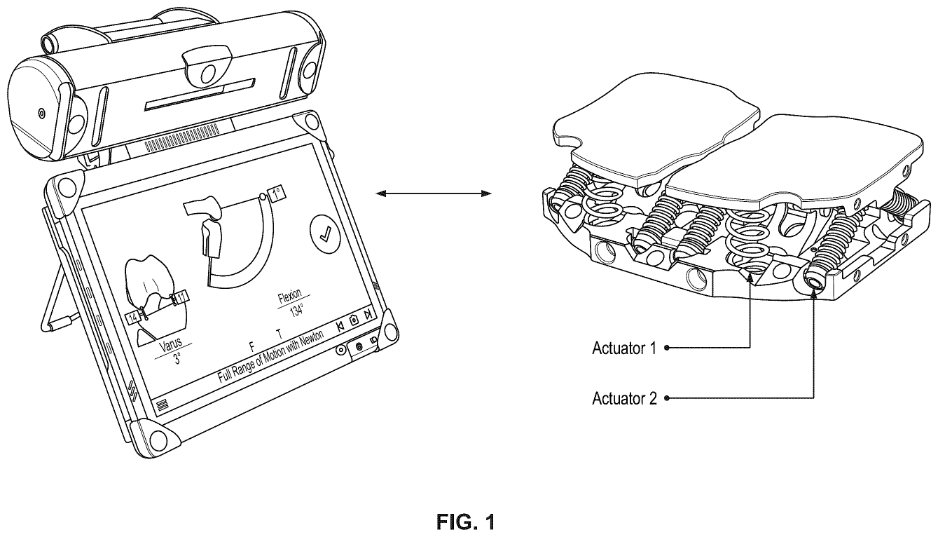

In at least some embodiments, the novel method to improve clinical outcomes may be applied to joint applications such as for example, knee joint applications using the CAOS system (as shown in FIGS. 1, 17 and 18). The CAOS system may provide surgeons with the ability to capture dynamic, 3D hip-knee-ankle (HKA) alignment at various stages of the total knee arthroplasty (TKA) procedure, both before any bone cuts or after a first bone cut may be performed (first arthroplasty stage) and after they may be fully executed (second arthroplasty stage).

FIG. 1 illustrates a platform combining a CAOS and an intra-articular tensioner device for facilitating an acquisition of the compartment-specific knee joint gaps throughout the full range of motion under force-controlled distraction maintained by two antagonist mechanical actuators. The intra-articular tensioner device may also be alternatively referred to as a distractor device, a distractor, a balancing device, and/or a ligament balancing device.

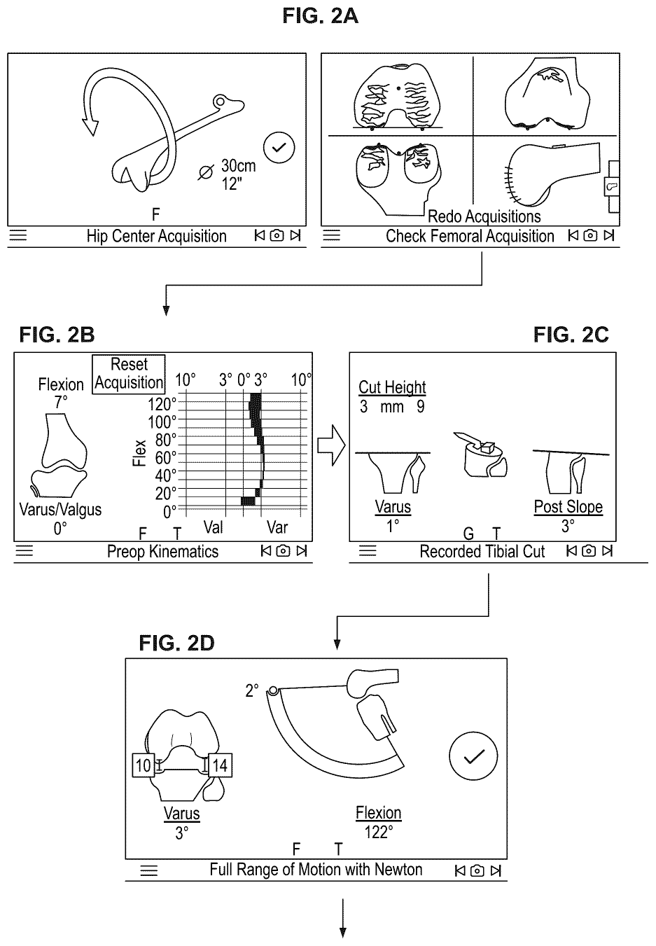

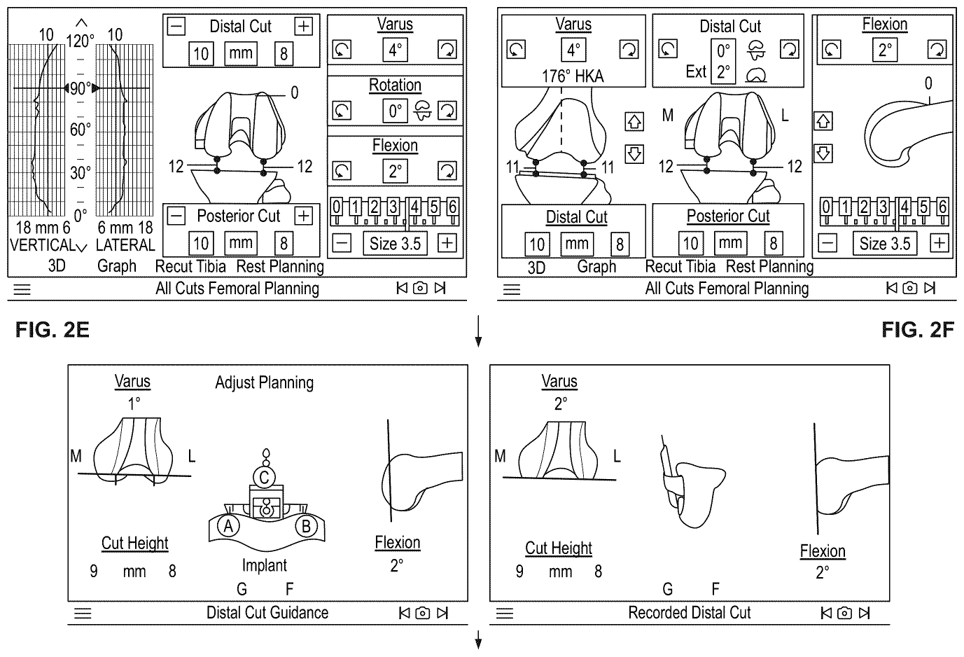

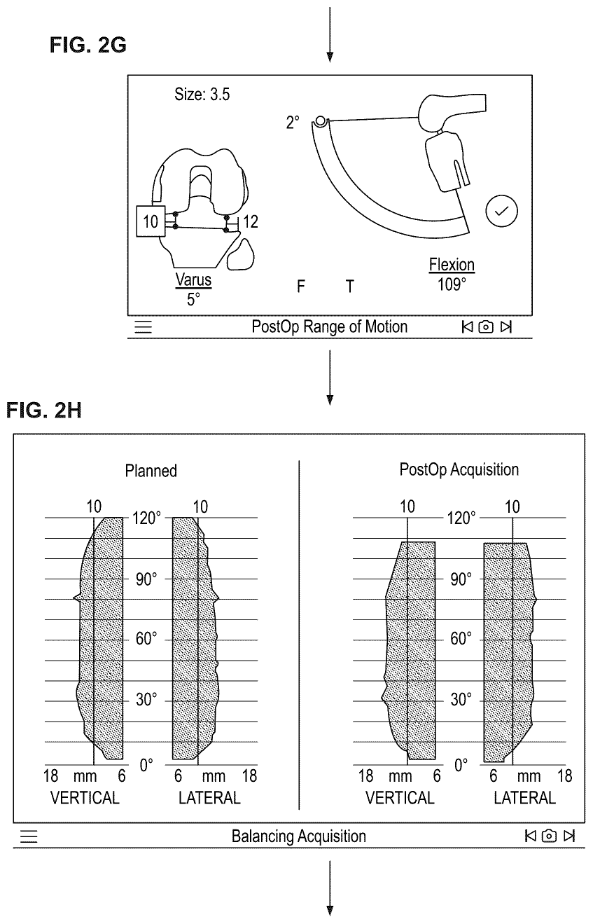

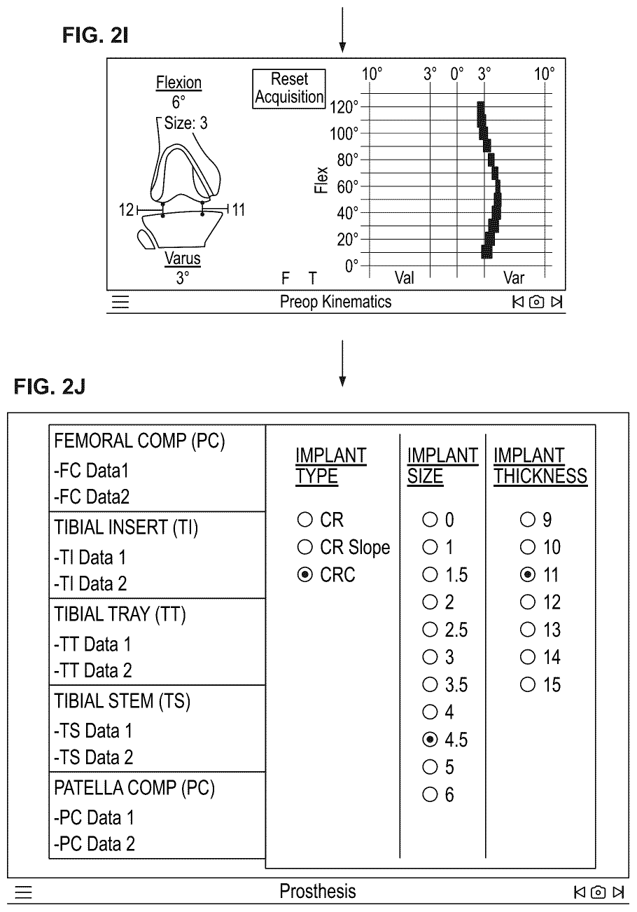

In at least some embodiments, a dataset may include 1087 TKA cases, following a tibia-first surgical workflow, that may be performed, for example, without limitation by 11 different surgeons. An illustration of the tibia-first workflow is shown in FIGS. 2A-2J.

Note that the embodiments of FIG. 1 are merely for conceptual and visual clarity and not by way of limitation of the embodiments described herein. Any CAOS technology may be used that allows for a 3D tracking of bone members. The same may be applied for the acquisition of the dHKA, which may be acquired through the CAOS by a manipulation of the joint in 3D (option 1) or may be acquired with an instrument, such as a tensioner or a distractor (e.g., intra-articular tensioner device) placed into the joint in order to tense the soft-tissue envelope during the manipulation of the joint in 3D (option 2). Please note that for option 2, there may be two additional sub-options:
Sub-option 2A: Before any cut, the intra-articular tensioner device should be thin enough to fit between the bone members at the level of the joint.
Sub-Option 2B: After a preliminary cut, an intra-articular tensioner device may be used to facilitate better access to the joint and to allow for the removal of osteophytes, such as for example, but not limited to the ligament balancing device described in U.S. Pat. No. 11,278,338, which is incorporated herein by reference in its entirety.

FIGS. 2A-2J are diagrams illustrating stages in a tibia-first surgical workflow in accordance with one or more embodiments of the present disclosure. FIG. 2A is a GUI screenshot illustrating an acquisition of anatomical landmarks. FIG. 2B is a GUI screenshot illustrating a pre-cut kinematics—3D HKA acquisition by manipulating the leg from extension to full flexion. FIG. 2C is a GUI screenshot illustrating a planning and resection of the proximal tibia. FIG. 2D is a GUI screenshot illustrating a pre-femoral cut acquisition using an intra-articular tensioner device. 3D HKA, medial, and lateral gaps may be captured by introducing a force-controlled intra-articular tensioner device into the joint space between the proximal tibia cut and the native femur, while the leg is neutrally manipulated from extension to flexion. FIG. 2E is a GUI screenshot illustrating a femoral planning based on alignment, size, and soft tissue considerations, establishing planned laxities. FIG. 2F is a GUI screenshot illustrating an execution of femoral cuts and placement of a trial femoral component on the distal femur. FIG. 2G is a GUI screenshot illustrating a post-femoral cut acquisition with the intra-articular tensioner device reintroduced into the joint space between the tibial cut and the trial component, capturing final medial gaps, lateral gaps, and 3D HKA. FIG. 2H is a GUI screenshot illustrating a verification of joint gaps by comparing planned and actual gaps. FIG. 2I is a GUI screenshot illustrating post-cut kinematics-3D HKA acquisition after all cuts. FIG. 2J is a GUI screenshot illustrating a final selection of implant parameters.

In at least some embodiments, active tracking arrays maybe initially attached to the femur and tibia. Anatomical landmarks may be registered (FIG. 2A) using the imageless CAOS system. To assess patient deformity and/or knee kinematics, the knee joint may be manipulated through its full range of flexion while the CAOS system may capture 3D HKA alignment. Varus and valgus (V&V) angles at specific flexion points may be displayed along the arc of flexion (FIG. 2B).

In at least some embodiments, a proximal tibial resection may be performed according to the surgeon's preference for resection thickness, varus/valgus orientation, and posterior slope (FIG. 2C). At this stage, the intra-articular device may be placed between the resected tibial surface and the native femur, applying a distraction force to maintain a quasi-constant distraction force in each compartment, regardless of joint gap variations, with the extensor mechanism in place. Subsequently, the knee may be moved through its range of motion, allowing the CAOS system to capture both medial and lateral joint gaps (FIG. 2D). This step may also provide updated 3D HKA measurements for the pre-femoral cut stage.

In at least some embodiments, with these inputs, femoral resection planning may be completed based on considerations of soft tissue balance, alignment, and/or component sizing (FIG. 2E). After the femoral cuts are made per the defined surgical plan, a trial femoral component may be positioned on the prepared distal femur (FIG. 2F). The intra-articular device may be reinserted into the joint space, now positioned between the tibial cut and the trial femoral component. The limb may then be manipulated from extension through full flexion, and the CAOS system may then capture the spatial positioning of the trial component relative to the tibial cut surface (FIG. 2G). A second set of joint gaps, or “checked gaps,” may be obtained and displayed alongside the initial “planned gaps” defined during the planning stage (FIG. 2H). Updated 3D HKA measurements for post-femoral cut stage may be available. Then, the limb may be manipulated without the intra-articular tensioner device from a full arc of motion, and final kinematics, after all cuts, may be acquired (FIG. 2I). The implant parameters may be selected for the surgery and displayed for review as the final step (FIG. 2J).

In at least some embodiments, with regard to dynamic acquisitions (i.e., 3D HKA), the CAOS system may enable the capture of dynamic acquisitions, such as 3D HKA, at several key stages in the procedure: pre-cut kinematics (FIG. 2B), pre-femoral cut intra-articular tensioner device acquisition (FIG. 2D), post-femoral cut intra-articular tensioner device acquisition (FIG. 2G), and/or post-cut kinematics (FIG. 2I). During the pre-cut and/or post-cut kinematics stages, surgeons may apply varus-valgus stress to the limb rather than neutral manipulation. In contrast, for intra-articular tensioner device acquisitions, neutral manipulation may be applied to capture joint gaps.

In at least some embodiments, the classification algorithm may process 3D HKA data from any of these four dynamic acquisition stages separately to generate dynamic knee phenotypes for each stage. For this analysis, data from the pre-femoral cut and/or post-femoral cut intra-articular tensioner device acquisitions may be used to develop the classification algorithms.

Note that the terms “pre-femoral cut intra-articular tensioner device acquisition” and “post-femoral cut intra-articular tensioner device acquisition” may be referred to herein as “pre-cut” and “post-cut,” respectively, with regard to the knee joint.

In at least some embodiments, a method for a dynamic knee alignment classification may include at least one of the two phases: 1) clustering and 2) evolution. At a higher level, a clustering phase may be applied to categorize the dynamic trajectories and then an evolution phase may be applied on the lower level to further refine the categorization at a more detailed level.

Note that the method for the dynamic knee alignment classification into a knee alignment classification phenotype may be performed using a joint phenotype classification algorithm for a joint, that may also be also referred to herein as a knee alignment classification algorithm, or a knee alignment algorithm for the knee joint.

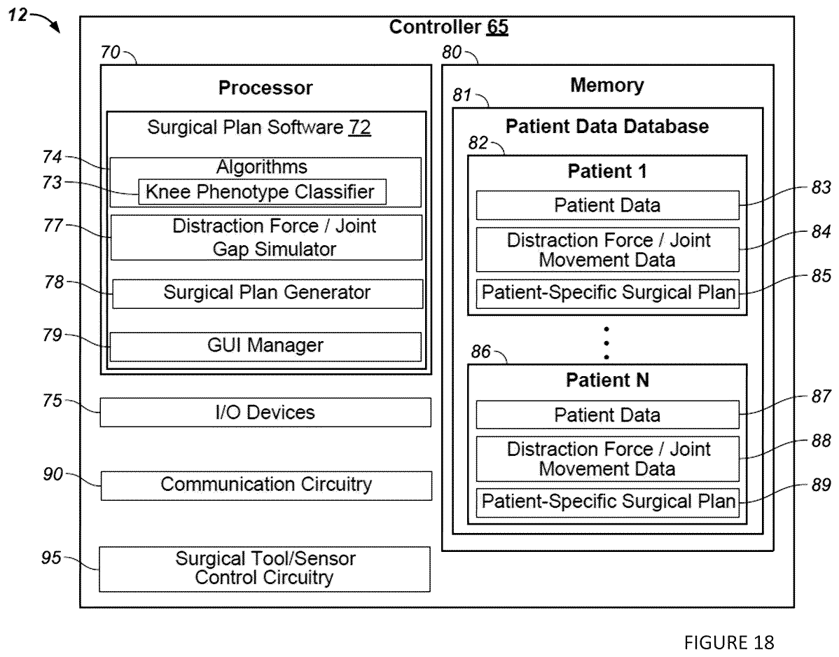

In at least some embodiments, the joint phenotype classification algorithm may include one computational module or a sequence of computational modules as described hereinbelow that may be executed by at least one processor of a computer-assisted orthopedic system to analyze intraoperative three-dimensional alignment data of a knee joint (e.g., as shown herein in FIG. 18). The joint phenotype classification algorithm may be configured to process data acquired at both first and second arthroplasty stages of a surgical procedure.

In at least some embodiments, the joint phenotype classification algorithm may include a feature extraction module that may select, from the 3D alignment data, at least one joint-specific metric, such as for example, at least one varus-valgus angle at a plurality of predetermined flexion angles of the knee joint for each of the first and second arthroplasty stages. The feature extraction module may utilize data extraction and interpolation techniques to ensure that the selected varus-valgus angles correspond to clinically relevant flexion points.

In at least some embodiments, the joint phenotype classification algorithm may include a feature vector construction module that may transform each set of selected joint-specific metrics, such as for example, selected varus-valgus angles for a knee joint into a corresponding feature vector in an N-dimensional feature space, where N is any integer that may represent the number of predetermined flexion angles and thus, a higher-dimensional feature space. This transformation may enable the subsequent computational analysis of dynamic knee alignment patterns.

In at least some embodiments, the joint phenotype classification algorithm may include an unsupervised clustering module that may apply a clustering algorithm, such as k-means, to the constructed feature vectors. This module may assign each feature vector to one of a plurality of clusters, thereby organizing the dynamic alignment data into groups that may represent distinct joint motion patterns, such as knee joint motion patterns.

In at least some embodiments, the joint phenotype classification algorithm may include a classification module that may classify, for each feature vector, an extension state and a flexion state as one of varus, neutral, or valgus ion the case of a knee joint. This classification may be based on the varus-valgus angle at a respective extension flexion angle and at one of the plurality of predetermined flexion angles, thereby generating an evolution class for each case.

In at least some embodiments, the joint phenotype classification algorithm may include a phenotype definition module that may combine each cluster assignment and the corresponding evolution class to define a dynamic knee alignment phenotype for each of the first and second arthroplasty stages. This combination may provide a comprehensive characterization of the patient's knee alignment behavior.

In at least some embodiments, the joint phenotype classification algorithm may include a comparison module that may compare the dynamic knee phenotype at the first arthroplasty stage with the dynamic knee phenotype at the second arthroplasty stage to determine a phenotype match or a phenotype transition therebetween. This comparison may enable the system to provide real-time feedback to the surgeon regarding the preservation or alteration of the patient's native alignment pattern.

In at least some embodiments, with regard to clustering, to simplify implementation, 3D HKA frontal alignment (VV angles at distinct flexion angles) measurements from both pre-cut and/or post-cut acquisitions may be considered for analysis. VV angles may be available at distinct flexion angles (0°, 5°, 10°, 15°, 20°, 30°, 45°, 60°, 75°, 90°, 105°, 120°). For features extraction, each 2D dynamic trajectory (VV and flexion) may be transformed into an N-dimensional VV features space (here N=12).

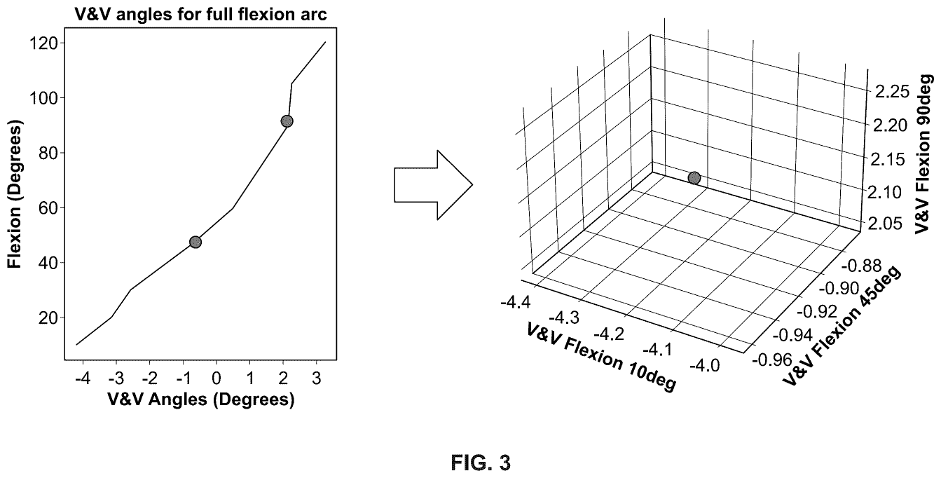

FIG. 3 are graphs illustrating a transformation of a 2D dynamic trajectory (VV and flexion) into a single point (e.g., a feature vector point) in a three-dimensional feature space in accordance with one or more embodiments of the present disclosure. This approach may be extended to the full 12-dimensional feature space. Once the data may be transformed, an unsupervised clustering method, such as k-means, may be applied to group the dataset into clusters, aiming to organize similar data points together and reveal underlying patterns within the data.

In at least some embodiments, the feature vector point may provide a means to convert complex, continuous, and multi-dimensional clinical data—specifically, the dynamic relationship between varus-valgus angles and flexion angles throughout the knee's range of motion—into a structured, quantitative representation that may be suitable for computational analysis. By transforming the two-dimensional dynamic trajectory of varus-valgus and flexion into a single feature vector point in a multi-dimensional feature space, the system may enable efficient storage, retrieval, and comparison of patient-specific alignment data by encapsulating the entire dynamic behavior of the knee joint across multiple flexion angles into a fixed-length, standardized numerical format.

In at least some embodiments, the feature vector point may facilitate the application of machine learning and clustering algorithms. Once the dynamic trajectory may be represented as a feature vector point, unsupervised clustering algorithms such as k-means, for example, may be applied. This may enable the system to automatically group similar knee kinematic patterns, identify clinically relevant phenotypes, and reveal underlying patterns in large datasets that may be difficult or impossible to discern manually.

In at least some embodiments, the use of feature vector points may allow for objective, reproducible, and automated classification of knee alignment phenotypes. This approach may reduce subjectivity and inter-operator variability that may occur with manual or visual assessment of dynamic trajectories, thereby supporting more consistent and reliable clinical outcomes.

In at least some embodiments, the approach of transforming dynamic trajectories into feature vector points may be scalable and extensible. The system may extend from three-dimensional to higher-dimensional feature spaces, such as a 12-dimensional space, for example, allowing for the incorporation of more detailed and granular data points. This may improve the accuracy and clinical relevance of the classification.

In at least some embodiments, the transformation into feature vector points may enable rapid computational processing, which may be essential for providing real-time intraoperative feedback to the medical practitioner. This capability may support personalized surgical planning and adjustment, thereby enhancing the ability to deliver individualized patient care.

In at least some embodiments, with regard to FIG. 3, which illustrates transforming a 2D dynamic trajectory (VV and flexion) into a single point in a three-dimensional feature space. While this process may be extended to 12-dimensional feature space (VV at all flexion angles), only a 3D scatter plot may be shown here merely for visual and conceptual clarity and not by way of limitation of the embodiments disclosed herein.

In at least some embodiments, the standard k-means training procedure may begin by selecting k initial centroids and initializing means for these k clusters. Data points may then be assigned to the nearest centroid, after which the means of each cluster may be recalculated. This process iteratively repeats until convergence criteria may be satisfied. The procedure may be executed across values of k from 1 to K, for example, and the Within-Cluster Sum of Squares (WCSS)—the sum of squared distances between each point and its cluster centroid—may be calculated for each value. The optimal number of clusters may be determined using the Elbow method, for example, supplemented by other evaluation metrics such as the Davies-Bouldin (DB) index, silhouette coefficient, and/or Calinski-Harabasz (CH) score to assess clustering performance and the selection of VV features.

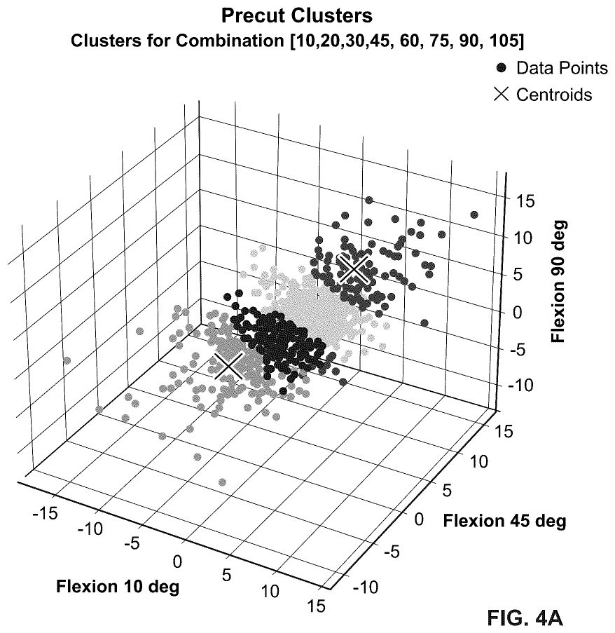

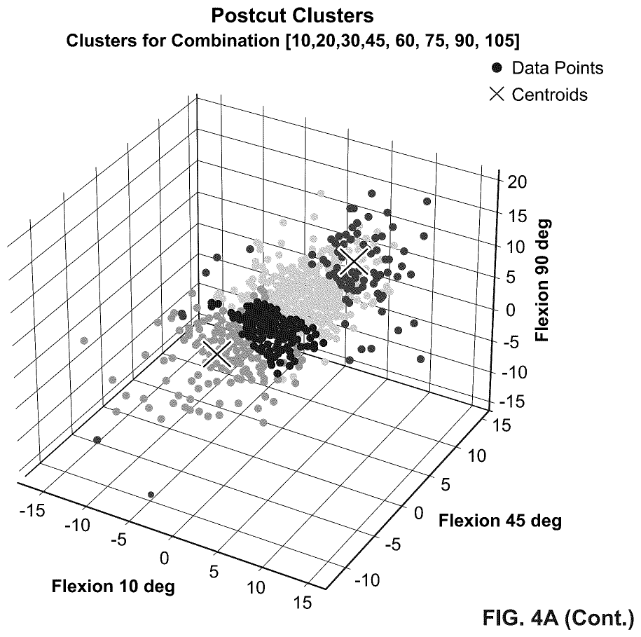

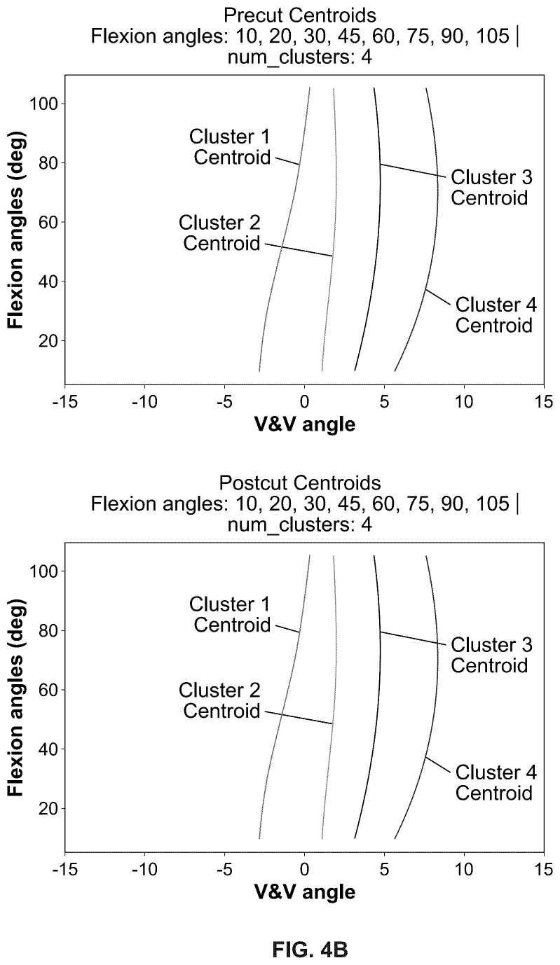

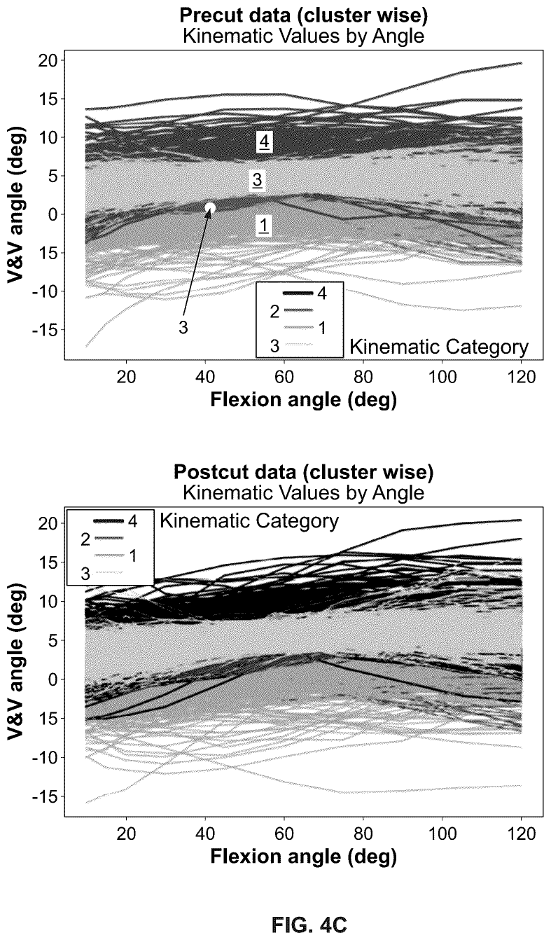

In at least some embodiments, the k-means method may be applied separately to pre-cut and post-cut data, yielding an optimal 8-dimensional feature space with VV angles at 10°, 15°, 20°, 30°, 45°, 60°, 75°, 90°, and 105° of flexion, and may result in four clusters for both pre-cut and post-cut data. These exemplary results are shown in FIGS. 4A-4C.

FIGS. 4A-4C are graphs illustrating clustering results for pre-cut and post-cut data using k-means with an optimal 8-dimensional feature space based on VV angles at specific flexion points (10°, 15°, 20°, 30°, 45°, 60°, 75°, 90°, and 105°) in accordance with one or more embodiments of the present disclosure.

FIG. 4A presents a 3D scatter plot of the four identified clusters, while FIGS. 4B and 4C illustrate the centroids of each cluster and the distribution of 1087 cases across the four clusters, marked by distinct colors for each cluster in accordance with one or more embodiments of the present disclosure.

In at least some embodiments with regard to FIGS. 4A-4C, the clustering method may effectively organize the data from 1087 cases into four distinct clusters, labeled 1 through 4. FIG. 4C may reveal that the VV dynamic trajectory may evolve from extension to flexion, showing non-linear patterns rather than horizontal lines. While clustering may successfully categorize the trajectories at a higher level, further categorization may be needed to fully capture their dynamic nature. To address this, a trajectory evolution method may be proposed to analyze VV angles at both extension and flexion, adding a finer categorization layer beyond the initial clustering.

In at least some embodiments with regard to evolution, the evolution method may focus on classifying the VV angle at two states: an extension state (10° flexion) and a flexion state (90° flexion). Each state may be categorized based on VV angle as follows:
if −3°≤VV≤3°, then state is neutral,
if VV>3°, then state is varus, and
if VV<−3°, then state is valgus.
   For each state, three possible categories may arise, resulting in nine potential class transitions from extension to flexion. Each class label may be expressed in the format: extension state to flexion state. The nine class labels may be, for example: neutral to neutral, neutral to varus, neutral to valgus, varus to neutral, varus to varus, varus to valgus, valgus to neutral, valgus to varus, and valgus to valgus.

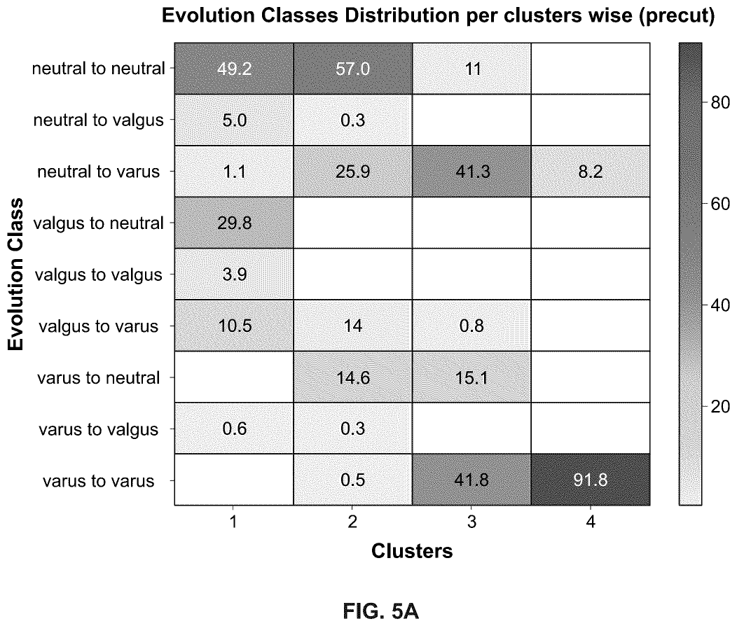

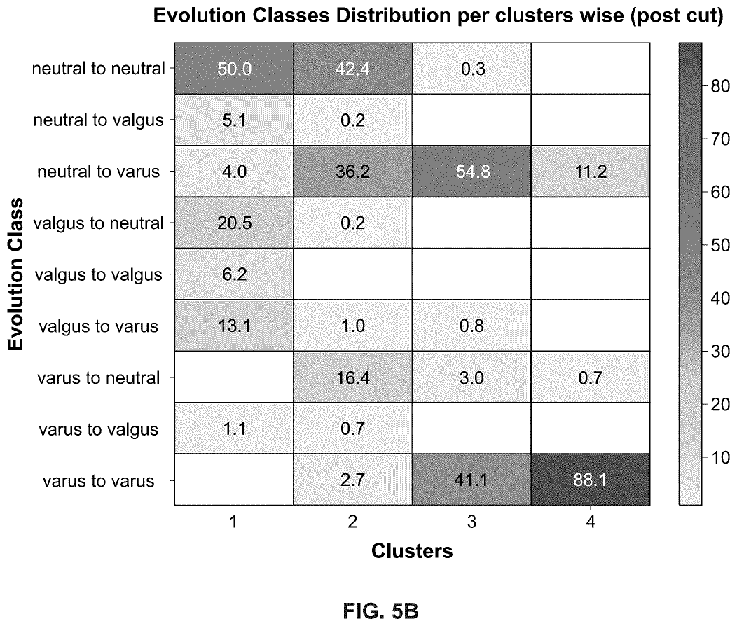

FIGS. 5A-5B are heat maps displaying the distribution of clusters and evolution classes for 1087 cases under pre-cut and post-cut conditions in accordance with one or more embodiments of the present disclosure. Clusters may be represented along the x-axis with evolution labels on the y-axis.

In at least some embodiments, the clustering and evolution classification method may be applied to the dataset of 1087 cases. The distribution of clusters and evolution classes may be presented as heat maps in FIGS. 5A-5B, with clusters on the x-axis and evolution labels on the y-axis for both pre-cut and post-cut conditions. In each column, the distribution of evolution classes within each cluster may be displayed, with values summing to 100%. As shown in FIGS. 5A-5B, among the 36 possible combinations (4 clusters×9 evolution classes), approximately one-third may include a sufficient percentage of cases.

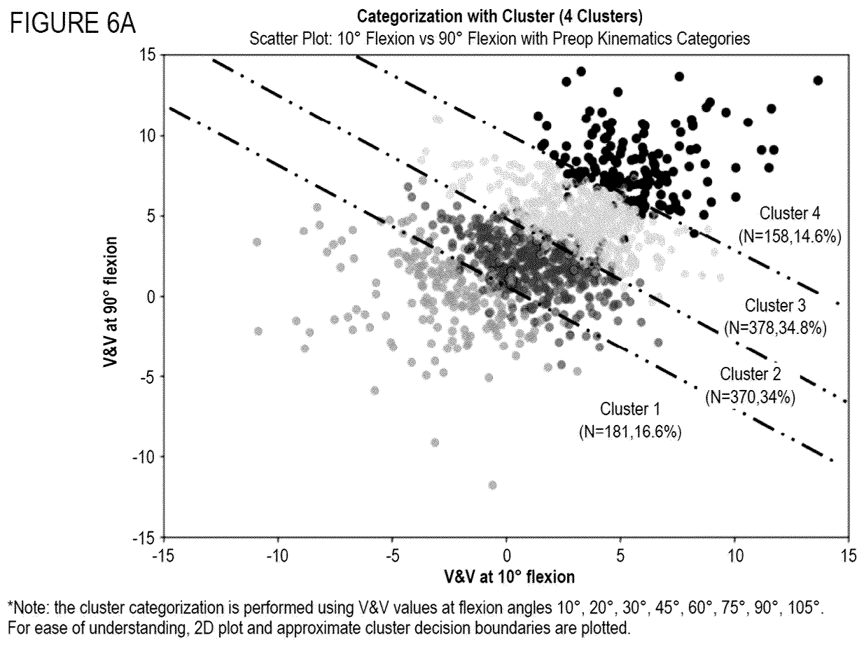

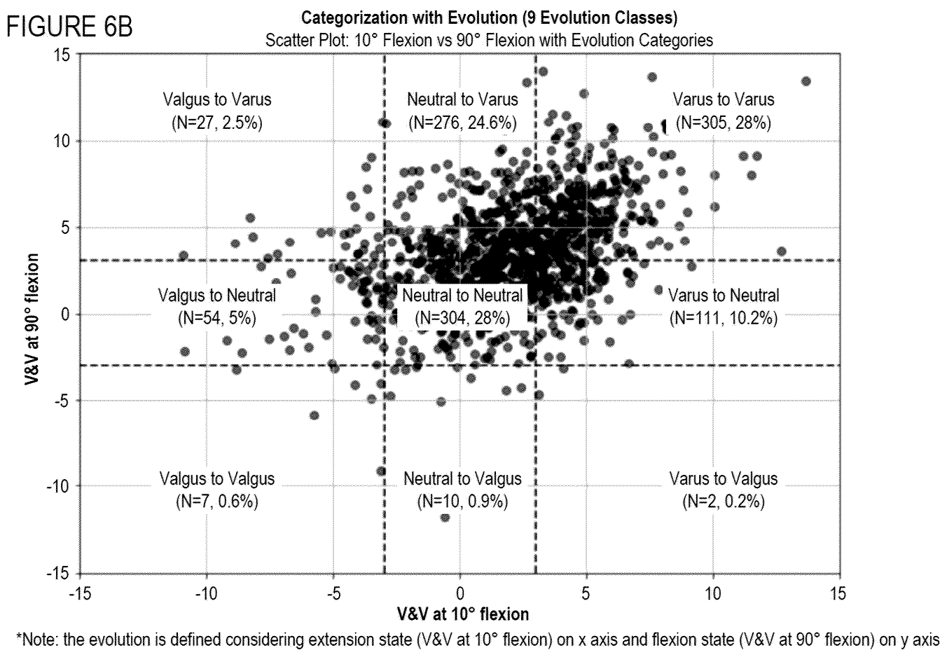

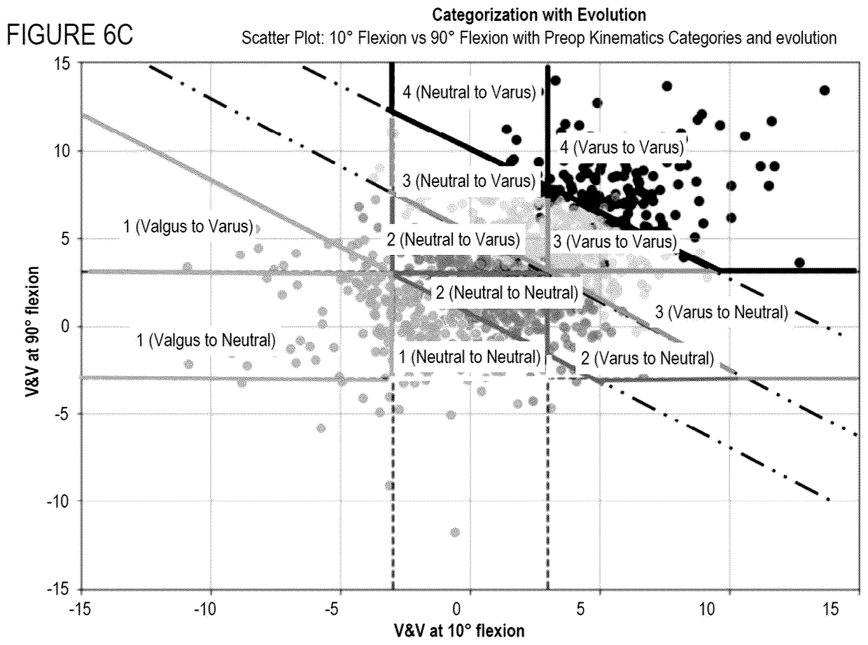

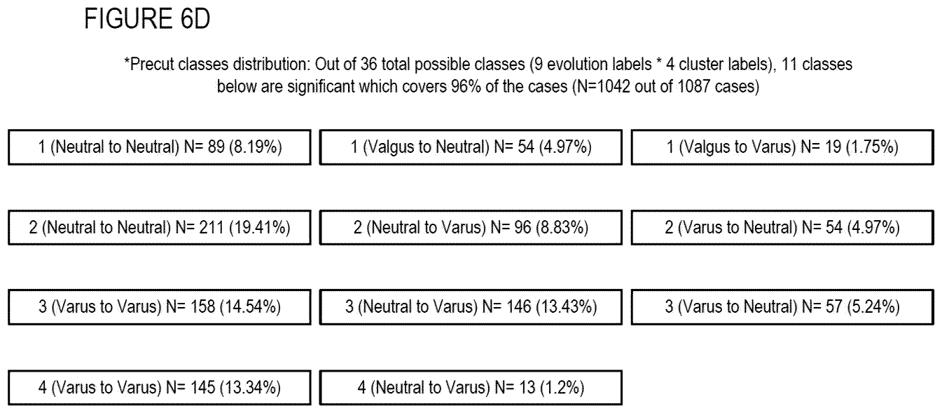

FIGS. 6A-6D are diagrams illustrating a 2D visualization of pre-cut kinematics conditions in accordance with one or more embodiments of the present disclosure. FIG. 6A illustrates a scatter plot with decision boundaries differentiating clusters in the 1087 cases, with each point representing a 2D dynamic VV trajectory at specific flexion angles. FIG. 6B illustrates an evolution class distribution with an extension state on the x-axis and flexion state on the y-axis. FIG. 6C may illustrate combined clustering and evolution categories, showing integrated classifications. FIG. 6D illustrates a summary of the 11 significant dynamic knee phenotypes, covering 96% of cases (1042/1087).

In at least some embodiments, pre-cut kinematics may be visualized in 2D in FIG. 6B. FIG. 6A shows a 2D scatter plot with approximate decision boundaries, illustrating the distinction between clusters within the 1087 cases. Each point in this plot may represent a 2D dynamic trajectory (VV trajectory at distinct flexion angles). While clustering was performed in 8-dimensional VV space, the data may be projected here in 2D for simplicity.

In at least some embodiments, FIG. 6B may categorize all 1087 cases by evolution class, with the extension state on the x-axis and flexion state on the y-axis. FIG. 6B displays the possibility of classifying 1087 cases into 9 classes only by evolution classification criteria. FIG. 6C may combine both clustering and evolution categories, illustrating modified decision boundaries to show integrated classifications. FIG. 6C displayed dominant 11 phenotypes out of total 36 possible phenotypes. In this representation, each dynamic knee phenotype may be labeled as Cluster (Evolution Class) or Cluster (Extension State to Flexion State). The method may theoretically allow for 36 unique dynamic knee phenotypes (from 4 clusters and 9 evolution classes). However, for this dataset, only 11 phenotypes may be significant, encompassing 96% of cases (1042 out of 1087), as illustrated in FIG. 6D.

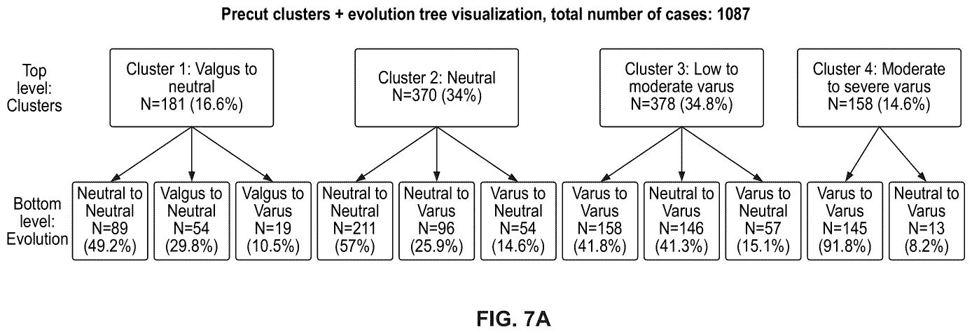

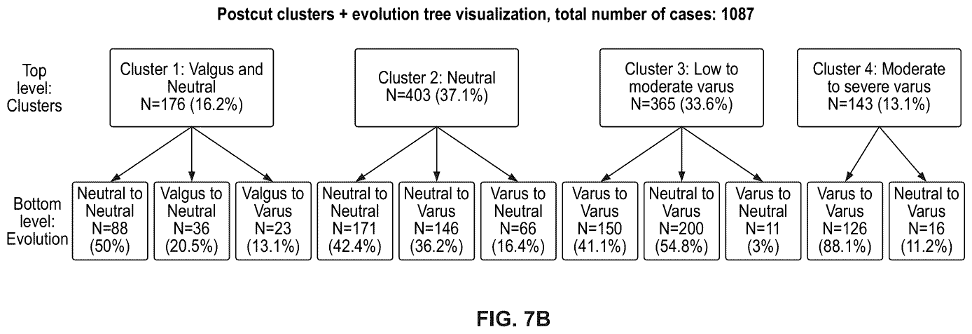

FIGS. 7A-7B are diagrams illustrating a tree visualization of the 11 dominant dynamic knee phenotypes, with cluster distributions shown at the top level and evolution distributions at the lower level for both pre-cut (FIG. 7A) and post-cut data (FIG. 7B) in accordance with one or more embodiments of the present disclosure.

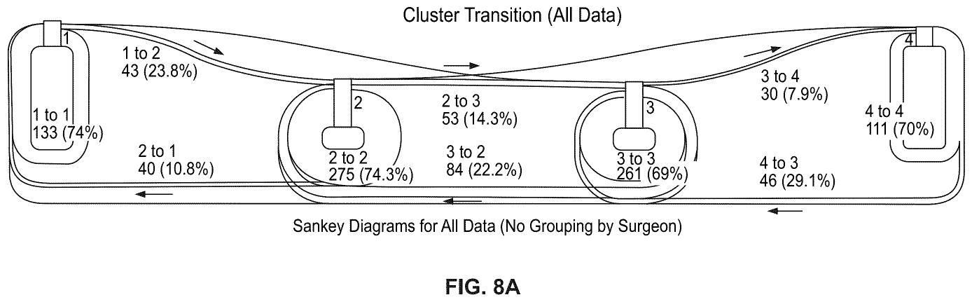

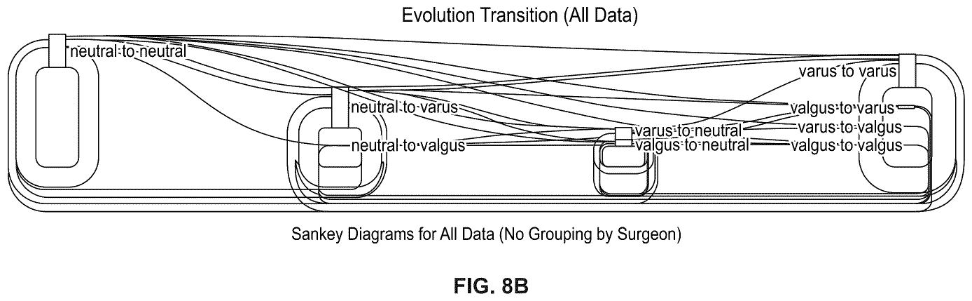

FIGS. 8A-8B are Sankey diagrams illustrating the transitions from pre-cut to post-cut dynamic knee phenotypes across 1087 cases performed by 11 surgeons in accordance with one or more embodiments of the present disclosure. FIG. 8A shows the transitions between clusters, while FIG. 8B displays the transitions at the more detailed evolution class level.

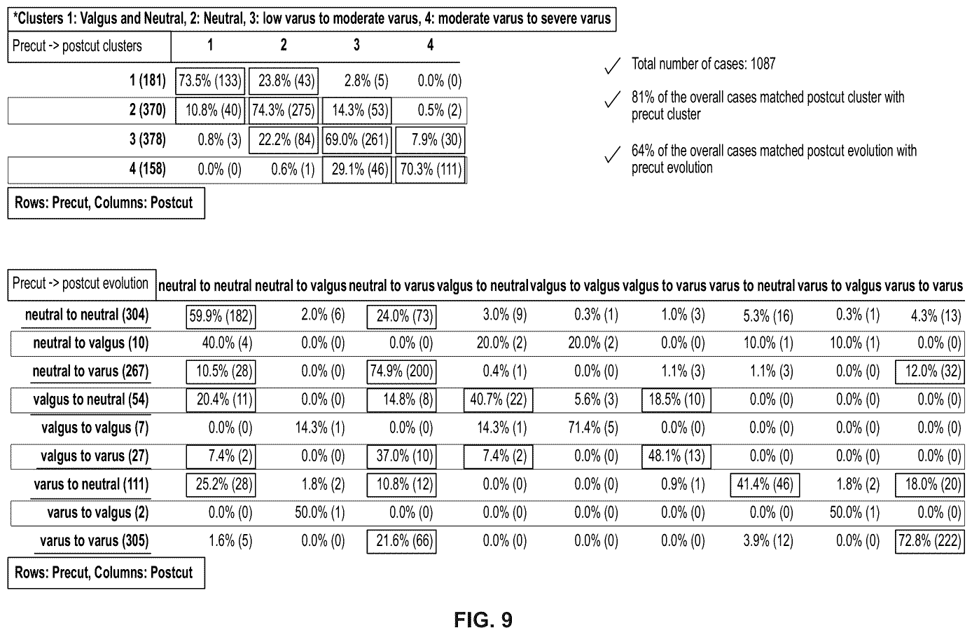

FIG. 9 are tables presenting pre-cut to post-cut dynamic knee phenotype transitions in tabular form, with cluster-level transitions (top) evolution-level transitions (bottom) in accordance with one or more embodiments of the present disclosure. The tables show that across all surgeons, clusters matched for 81% of cases, while evolution class transitions were consistent for 64% of cases from pre-cut to post-cut. The highlighted boxes in the tables infer noticeable transitions from precut phenotypes to postcut phenotypes.

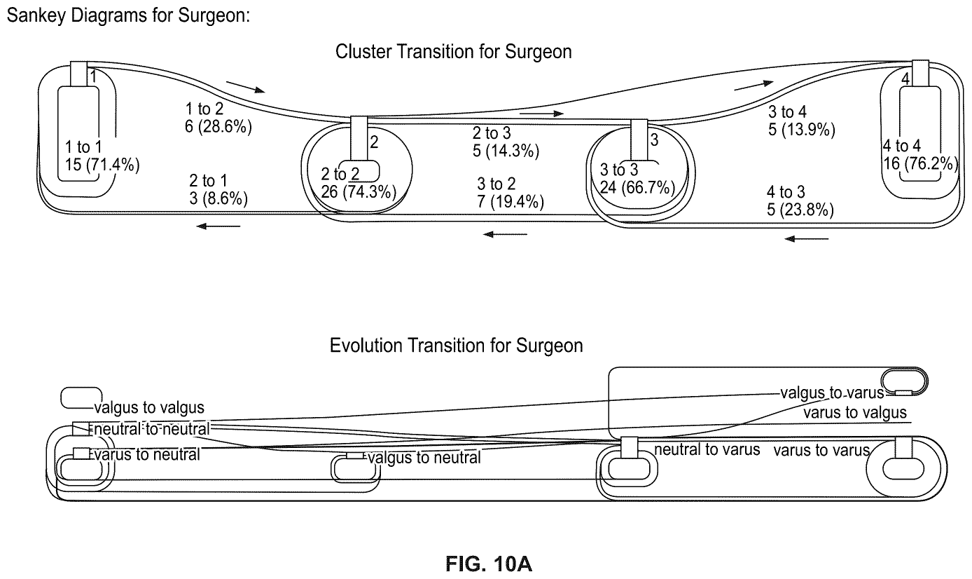

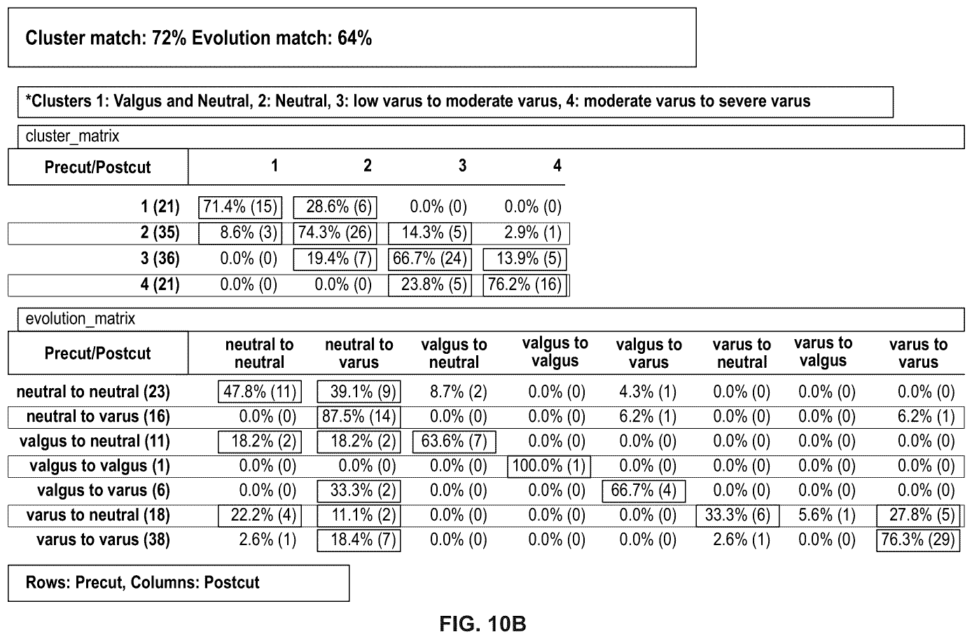

FIGS. 10A-10B illustrates diagrams that highlight the transitions for an exemplary high-volume Surgeon A, that performed 113 cases, using both Sankey diagrams and transition tables, in accordance with one or more embodiments of the present disclosure. Surgeon A's match rates from pre-cut to post-cut were 72% at the cluster level and 64% at the evolution level. The highlighted boxes in the tables infer noticeable transitions from precut phenotypes to postcut phenotypes.

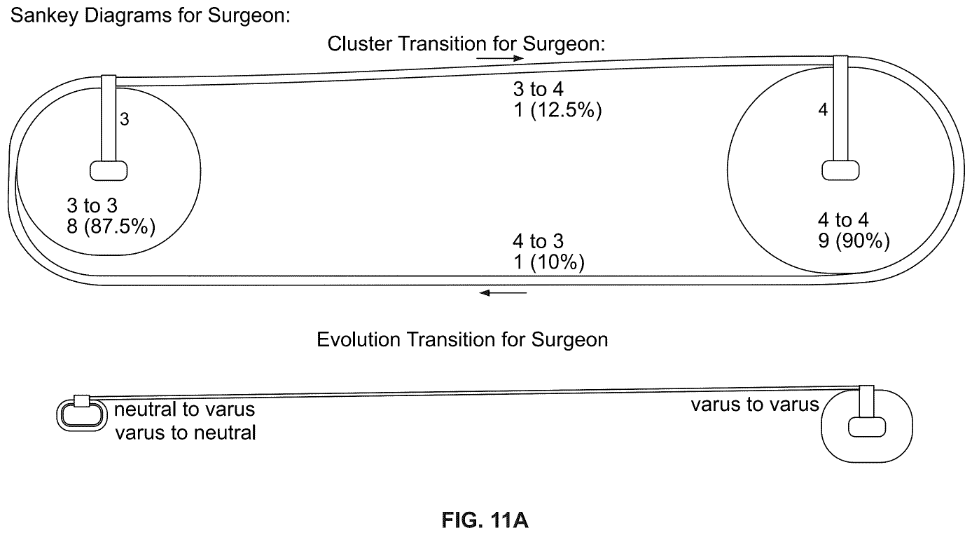

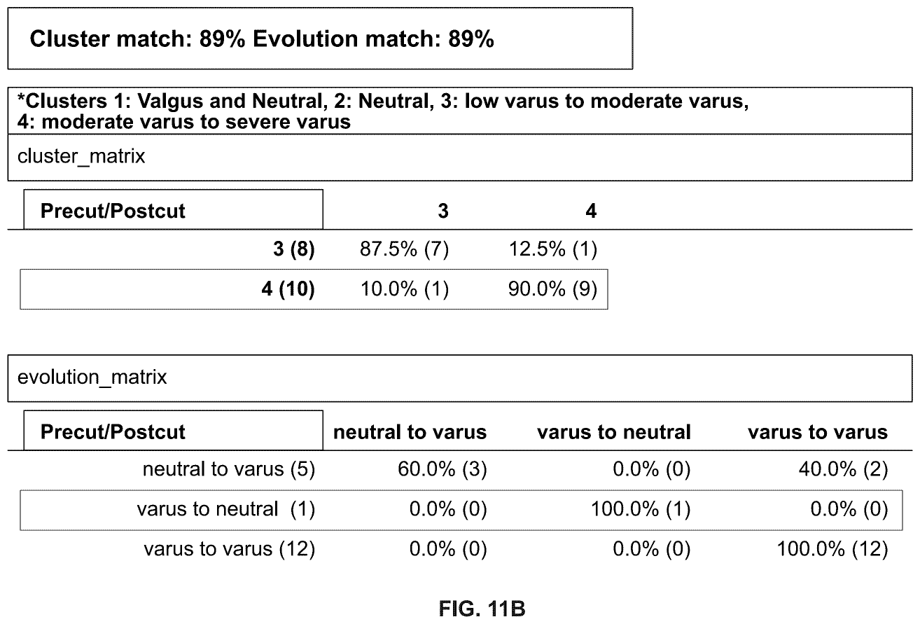

FIGS. 11A-11B illustrates transitions for a low-volume Surgeon B (who handled 18 cases) with Sankey diagrams and transition tables in accordance with one or more embodiments of the present disclosure. Surgeon B achieved a match rate of 89% at both the cluster and evolution levels. Notably, this exemplary surgeon had no cases in clusters 1 and 2, suggesting a focus on varus and neutral trajectories among the patients operated on.

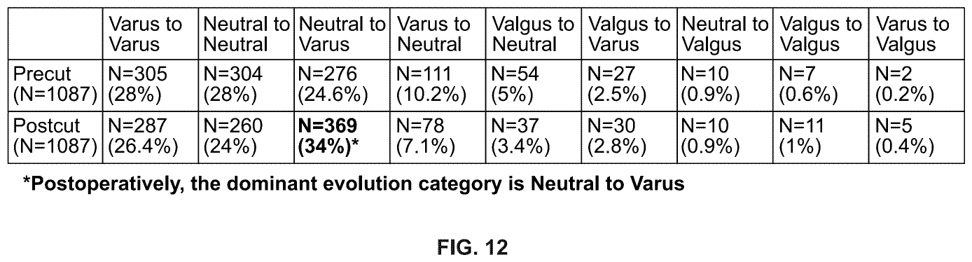

FIG. 12 is a table displaying further details of the distribution of evolution classes for both the pre-cut and post-cut conditions in accordance with one or more embodiments of the present disclosure.

In at least some embodiments, exemplary inventive, specially programmed computing systems/platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk(TM), TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. In at least some embodiments, the NFC can represent a short-range wireless communications technology in which NFC-enabled devices are “swiped,” “bumped,” “tap” or otherwise moved in close proximity to communicate. In at least some embodiments, the NFC could include a set of short-range wireless technologies, typically requiring a distance of 10 cm or less. In at least some embodiments, the NFC may operate at 13.56 MHz on ISO/IEC 18000-3 air interface and at rates ranging from 106 kbit/s to 424 kbit/s. In at least some embodiments, the NFC can involve an initiator and a target; the initiator actively generates an RF field that can power a passive target. In at least some embodiments, this can enable NFC targets to take very simple form factors such as tags, stickers, key fobs, or cards that do not require batteries. In at least some embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In at least some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In at least some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In at least some embodiments, as detailed herein, one or more of exemplary inventive computer-based systems/platforms, exemplary inventive computer-based devices, and/or exemplary inventive computer-based components of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a social media post, a map, an entire application (e.g., a calculator), etc. In at least some embodiments, as detailed herein, one or more of exemplary inventive computer-based systems/platforms, exemplary inventive computer-based devices, and/or exemplary inventive computer-based components of the present disclosure may be implemented across one or more of various computer platforms such as, but not limited to: (1) FreeBSD, NetBSD, OpenBSD; (2) Linux; (3) Microsoft Windows; (4) OS X (MacOS); (5) MacOS 11; (6) Solaris; (7) Android; (8) iOS; (9) Embedded Linux; (10) Tizen; (11) WebOS; (12) IBM i; (13) IBM AIX; (14) Binary Runtime Environment for Wireless (BREW); (15) Cocoa (API); (16) Cocoa Touch; (17) Java Platforms; (18) JavaFX; (19) JavaFX Mobile; (20) Microsoft DirectX; (21) . NET Framework; (22) Silverlight; (23) Open Web Platform; (24) Oracle Database; (25) Qt; (26) Eclipse Rich Client Platform; (27) SAP NetWeaver; (28) Smartface; and/or (29) Windows Runtime.

In at least some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In at least some embodiments, exemplary inventive computer-based systems/platforms, exemplary inventive computer-based devices, and/or exemplary inventive computer-based components of the present disclosure may be configured to handle numerous concurrent users that may be, but is not limited to, at least 100 (e.g., but not limited to, 100-999), at least 1,000 (e.g., but not limited to, 1,000-9,999) , at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In at least some embodiments, exemplary inventive computer-based systems/platforms, exemplary inventive computer-based devices, and/or exemplary inventive computer-based components of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, and/or objects. For example, such projections may be a visual overlay for a mobile augmented reality (MAR) application.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, the terms “proximity detection,” “locating,” “location data,” “location information,” and “location tracking” refer to any form of location tracking technology or locating method that can be used to provide a location of, for example, a particular computing device/system/platform of the present disclosure and/or any associated computing devices, based at least in part on one or more of the following techniques/devices, without limitation: accelerometer(s), gyroscope(s), Global Positioning Systems (GPS); GPS accessed using Bluetooth™; GPS accessed using any reasonable form of wireless and/or non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

As used herein, the terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In at least some embodiments, the exemplary inventive computer-based systems/platforms, the exemplary inventive computer-based devices, and/or the exemplary inventive computer-based components of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

The aforementioned examples are, of course, illustrative and not restrictive.

As used herein, the term “user” shall have a meaning of at least one user. In at least some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session or can refer to an automated software application which receives the data and stores or processes the data.

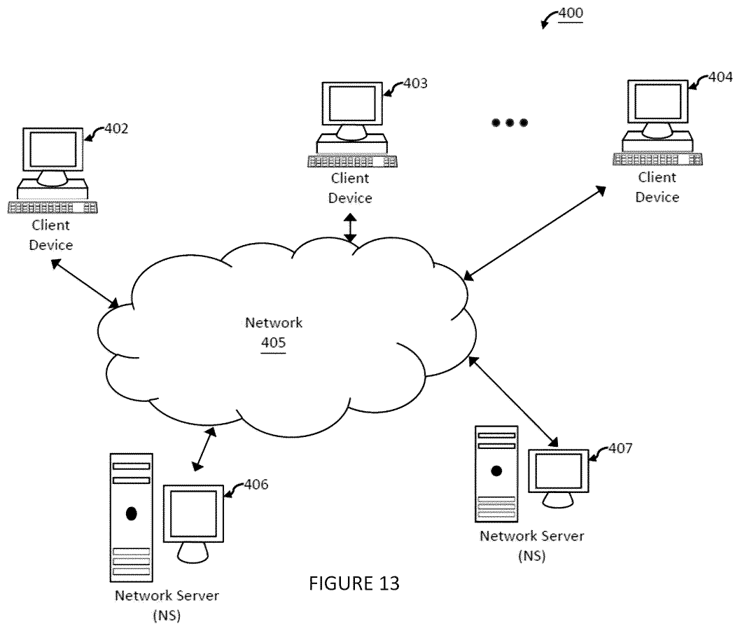

FIG. 13 depicts a block diagram of an exemplary computer-based system/platform 400 in accordance with one or more embodiments of the present disclosure. However, not all of these components may be required to practice one or more embodiments, and variations in the arrangement and type of the components may be made without departing from the spirit or scope of various embodiments of the present disclosure. In at least some embodiments, the exemplary inventive computing devices and/or the exemplary inventive computing components of the exemplary computer-based system/platform 400 may be configured to manage a large number of members and/or concurrent transactions, as detailed herein. In at least some embodiments, the exemplary computer-based system/platform 400 may be based on a scalable computer and/or network architecture that incorporates varies strategies for assessing the data, caching, searching, and/or database connection pooling. An example of the scalable architecture is an architecture that is capable of operating multiple servers.

In at least some embodiments, referring to FIG. 13, members 402-404 (e.g., clients) of the exemplary computer-based system/platform 400 may include virtually any computing device capable of receiving and sending a message over a network (e.g., cloud network), such as network 405, to and from another computing device, such as servers 406 and 407, each other, and the like. In at least some embodiments, the member devices 402-404 may be personal computers, multiprocessor systems, microprocessor-based or programmable consumer electronics, network PCs, and the like. In at least some embodiments, one or more member devices within member devices 402-404 may include computing devices that typically connect using a wireless communications medium such as cell phones, smart phones, pagers, walkie talkies, radio frequency (RF) devices, infrared (IR) devices, CBs, integrated devices combining one or more of the preceding devices, or virtually any mobile computing device, and the like. In at least some embodiments, one or more member devices within member devices 402-404 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, satellite, ZigBee, etc.). In at least some embodiments, one or more member devices within member devices 402-404 may include may run one or more applications, such as Internet browsers, mobile applications, voice calls, video games, videoconferencing, and email, among others. In at least some embodiments, one or more member devices within member devices 402-404 may be configured to receive and to send web pages, and the like. In at least some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In at least some embodiments, a member device within member devices 402-404 may be specifically programmed by either Java, .Net, QT, C, C++ and/or other suitable programming language. In at least some embodiments, one or more member devices within member devices 402-404 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

In at least some embodiments, the exemplary network 405 may provide network access, data transport and/or other services to any computing device coupled to it. In at least some embodiments, the exemplary network 405 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In at least some embodiments, the exemplary network 405 may implement one or more of a GSM architecture, a General Packet Radio Service (GPRS) architecture, a Universal Mobile Telecommunications System (UMTS) architecture, and an evolution of UMTS referred to as Long Term Evolution (LTE). In at least some embodiments, the exemplary network 405 may include and implement, as an alternative or in conjunction with one or more of the above, a WiMAX architecture defined by the WiMAX forum. In at least some embodiments and, optionally, in combination of any embodiment described above or below, the exemplary network 405 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In at least some embodiments and, optionally, in combination of any embodiment described above or below, at least one computer network communication over the exemplary network 405 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In at least some embodiments, the exemplary network 405 may also include mass storage, such as network attached storage (NAS), a storage area network (SAN), a content delivery network (CDN) or other forms of computer or machine readable media.

In at least some embodiments, the exemplary server 406 or the exemplary server 407 may be a web server (or a series of servers) running a network operating system, examples of which may include but are not limited to Microsoft Windows Server, Novell NetWare, or Linux. In at least some embodiments, the exemplary server 406 or the exemplary server 407 may be used for and/or provide cloud and/or network computing. Although not shown in FIG. 13, in at least some embodiments, the exemplary server 406 or the exemplary server 407 may have connections to external systems like email, SMS messaging, text messaging, ad content providers, etc. Any of the features of the exemplary server 406 may be also implemented in the exemplary server 407 and vice versa.

In at least some embodiments, one or more of the exemplary servers 406 and 407 may be specifically programmed to perform, in non-limiting example, as authentication servers, search servers, email servers, social networking services servers, SMS servers, IM servers, MMS servers, exchange servers, photo-sharing services servers, advertisement providing servers, financial/banking-related services servers, travel services servers, or any similarly suitable service-base servers for users of the member computing devices 401-404.

In at least some embodiments and, optionally, in combination of any embodiment described above or below, for example, one or more exemplary computing member devices 402-404, the exemplary server 406, and/or the exemplary server 407 may include a specifically programmed software module that may be configured to send, process, and receive information using a scripting language, a remote procedure call, an email, a tweet, Short Message Service (SMS), Multimedia Message Service (MMS), instant messaging (IM), internet relay chat (IRC), mIRC, Jabber, an application programming interface, Simple Object Access Protocol (SOAP) methods, Common Object Request Broker Architecture (CORBA), HTTP (Hypertext Transfer Protocol), REST (Representational State Transfer), or any combination thereof.

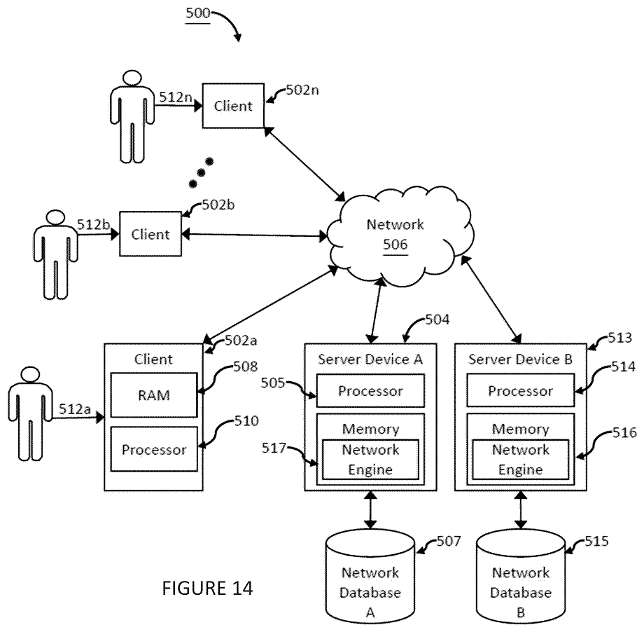

FIG. 14 depicts a block diagram of another exemplary computer-based system/platform 500 in accordance with one or more embodiments of the present disclosure. However, not all of these components may be required to practice one or more embodiments, and variations in the arrangement and type of the components may be made without departing from the spirit or scope of various embodiments of the present disclosure. In at least some embodiments, the member computing devices 502a, 502b thru 502n shown each at least includes a computer-readable medium, such as a random-access memory (RAM) 508 coupled to a processor 510 or FLASH memory. In at least some embodiments, the processor 510 may execute computer-executable program instructions stored in memory 508. In at least some embodiments, the processor 510 may include a microprocessor, an ASIC, and/or a state machine. In at least some embodiments, the processor 510 may include, or may be in communication with, media, for example computer-readable media, which stores instructions that, when executed by the processor 510, may cause the processor 510 to perform one or more steps described herein. In at least some embodiments, examples of computer-readable media may include, but are not limited to, an electronic, optical, magnetic, or other storage or transmission device capable of providing a processor, such as the processor 510 of client 502a, with computer-readable instructions. In at least some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In at least some embodiments, the instructions may comprise code from any computer-programming language, including, for example, C, C++, Visual Basic, Java, Python, Perl, JavaScript, and etc.

In at least some embodiments, member computing devices 502a through 502n may also comprise a number of external or internal devices such as a mouse, a CD-ROM, DVD, a physical or virtual keyboard, a display, a speaker, or other input or output devices. In at least some embodiments, examples of member computing devices 502a through 502n (e.g., clients) may be any type of processor-based platforms that are connected to a network 506 such as, without limitation, personal computers, digital assistants, personal digital assistants, smart phones, pagers, digital tablets, laptop computers, Internet appliances, and other processor-based devices. In at least some embodiments, member computing devices 502a through 502n may be specifically programmed with one or more application programs in accordance with one or more principles/methodologies detailed herein. In at least some embodiments, member computing devices 502a through 502n may operate on any operating system capable of supporting a browser or browser-enabled application, such as Microsoft™, Windows™, and/or Linux. In at least some embodiments, member computing devices 502a through 502n shown may include, for example, personal computers executing a browser application program such as Microsoft Corporation's Internet Explorer™, Apple Computer, Inc.'s Safari™, Mozilla Firefox, and/or Opera. In at least some embodiments, through the member computing client devices 502a through 502n, users, 512a through 512n, may communicate over the exemplary network 506 with each other and/or with other systems and/or devices coupled to the network 506. As shown in FIG. 10, exemplary server devices 504 and 513 may be also coupled to the network 506. In at least some embodiments, one or more member computing devices 502a through 502n may be mobile clients.

In at least some embodiments, at least one database of exemplary databases 507 and 515 may be any type of database, including a database managed by a database management system (DBMS). In at least some embodiments, an exemplary DBMS-managed database may be specifically programmed as an engine that controls organization, storage, management, and/or retrieval of data in the respective database. In at least some embodiments, the exemplary DBMS-managed database may be specifically programmed to provide the ability to query, backup and replicate, enforce rules, provide security, compute, perform change and access logging, and/or automate optimization. In at least some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In at least some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In at least some embodiments, the exemplary DBMS-managed database may be specifically programmed to include metadata about the data that is stored.

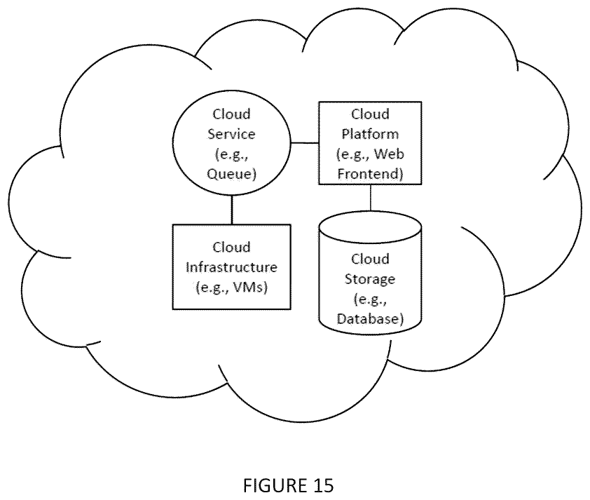

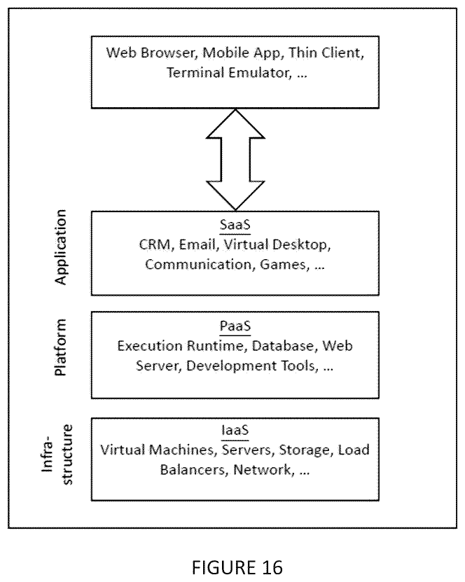

In at least some embodiments, the exemplary inventive computer-based systems/platforms, the exemplary inventive computer-based devices, and/or the exemplary inventive computer-based components of the present disclosure may be specifically configured to operate in an cloud computing/architecture such as, but not limiting to: infrastructure a service (IaaS), platform as a service (PaaS), and/or software as a service (Saas). FIGS. 15 and 16 illustrate schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

In at least some embodiments, the exemplary inventive computer-based systems/platforms, the exemplary inventive computer-based devices, and/or the exemplary inventive computer-based components of the present disclosure may be configured to utilize one or more exemplary AI/machine learning techniques applied to any of the algorithms 74 as described herein may be chosen from, but not limited to, decision trees, boosting, support-vector machines, neural networks, nearest neighbor algorithms, Naive Bayes, bagging, random forests, and the like. In at least some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In at least some embodiments and, optionally, in combination of any embodiment described above or below, an exemplary implementation of Neural Network may be executed as follows:
i) Define Neural Network architecture/model,
ii) Transfer the input data to the exemplary neural network model,
iii) Train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In at least some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In at least some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In at least some embodiments and, optionally, in combination of any embodiment described above or below, the exemplary aggregation function may be a mathematical function that combines (e.g., sum, product, etc.) input signals to the node. In at least some embodiments and, optionally, in combination of any embodiment described above or below, an output of the exemplary aggregation function may be used as input to the exemplary activation function. In at least some embodiments and, optionally, in combination of any embodiment described above or below, the bias may be a constant value or function that may be used by the aggregation function and/or the activation function to make the node more or less likely to be activated.

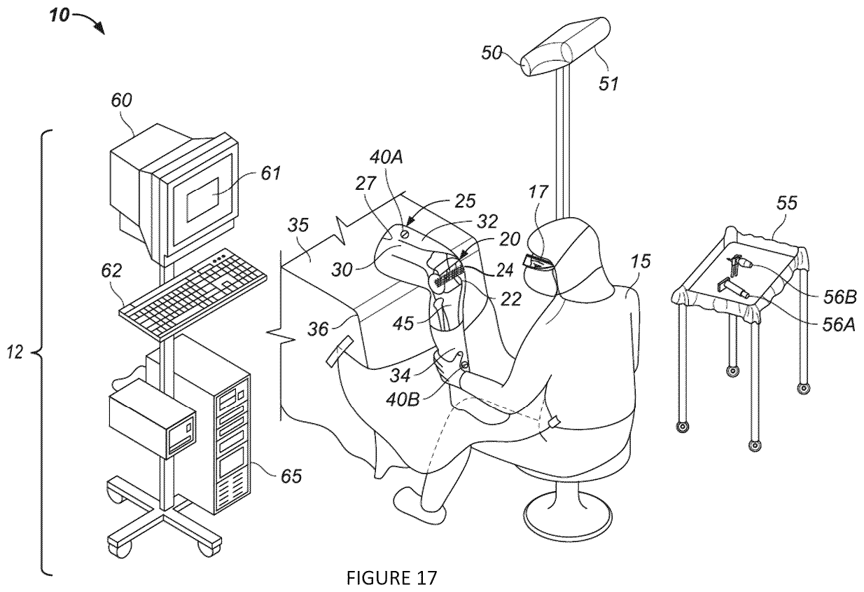

FIG. 17 schematically illustrates an operating room 10 using an improved computer-based surgery assistance device 12 (e.g., the CAOS system) for classification of total joint arthroplasty patient alignment based on intraoperative data acquisitions in accordance with one or more embodiments of the present disclosure. The embodiments shown in FIG. 17 refer to a total knee arthroplasty procedure. FIG. 17 shows a surgeon 15 operating on a leg 25 of a patient positioned on an operating table 35. The leg 25 of the patient may be placed through a surgical drape opening 27 for access to the leg 25 by the surgeon 15. In this exemplary embodiment, the surgeon 15 may perform a total knee arthroplasty procedure on the patient via an incision 22 made by the surgeon 15 to expose a knee joint 20 of the patient. A distractor 24 (e.g., such as the intra-articular tensioner device of FIG. 1) may be placed by the surgeon 15 into the knee joint 20 for soft tissue characterization under an application of at least one distraction force. The leg 25 as shown in FIG. 17 may include an upper portion 32 (e.g., a first member-thigh) with a femur 30 (e.g., first bone member), a lower portion 34 (e.g., a second member-calf) with a tibia 45 (e.g., second bone member), and the knee joint 20.

In at least some embodiments, at least one first tracking device 40A may be coupled to the upper portion 32 of the leg 25 (e.g., a first bone member) and at least one second tracking device 40B may be coupled to the lower portion 34 of the leg 25 (e.g., a second bone member). In other embodiments, the at least one first tracker 40A and the at least one second tracker 40B may be rigidly mounted to the bone members (e.g., respectively to the femur 30 and to the tibia 45 for the embodiments of FIG. 17). In other embodiments, any suitable configuration of trackers may be applied to the patient leg.

In at least some embodiments, the operating room 10 may include at least one imaging camera 50 shown schematically in FIG. 17 mounted on an image camera assembly 51. Note that any suitable number of cameras of any suitable type may be mounted on the image camera assembly 51 that may be used to track 3D objects. The at least one imaging camera 50 may be used to acquire a position and/or orientation of the bone members in a three-dimensional (3D) environment.

In at least some embodiments, the operating room 10 may include at least one surgical tool 56A and/or at least one surgical probe 56B placed on a cart 55 easily accessible by the surgeon 15 during the joint arthroplasty procedure.

In at least some embodiments, the surgery assistance device 12 in the operating room 10 may include a controller 65, a keyboard 62 and/or a display 60 displaying a graphic user interface (GUI) 61 with graphic user interface elements to allow the surgeon 15 to interact with.

In at least some embodiments, the display 60 may be a screen/monitor directly accessible to the surgeon 15 and/or by a wearable display 17 (e.g., heads up display, smart glasses) directly worn by the surgeon 15 during the surgical procedure to provide a computer-controlled augmented reality view for the surgeon 15. The controller 65 may be communicatively coupled to any of the surgical tools used by the surgeon 15 to perform the total joint arthroplasty.

In at least some embodiments, the controller 65 may display on the GUI 61 of the display 60 (e.g., the display of the CAOS system of FIG. 1 and the GUI screenshots of FIGS. 2A-2J), a patient-specific surgical plan to assist the surgeon 15 to perform the placement of the joint implant into the joint of the patient undergoing the total joint arthroplasty. The keyboard 62 may be used by the surgeon 15 or any other medical personnel assisting the surgeon 15 to input patient-specific data into the controller 65 via the keyboard 62 either before and/or during the joint arthroplasty procedure such that the algorithms executed by the controller 65 may generate and/or update the surgical plan in real time so as to assist the surgeon 15 before and/or during the joint arthroplasty procedure. The GUI 61 may further allow the surgeon 15 to vary the distraction force regimes to view the impact of changes in the soft tissue balancing of the joint for proper pre-operative and/or intra-operative surgical planning.

In at least some embodiments, the controller 65 (e.g., the I/O devices 92) may be configured to receive voice control commands and/or the display unit 60 may have touchscreen capabilities as an alternative to using the keyboard 62, where the surgeon 15 may use a pointer device, (e.g., an input device 92), for example, to activate the graphical user interface elements on the GUI 61 that are programmed to allow the surgeon 15 to adjust surgical parameters via the display unit 60 during the surgical procedure, as will be shown hereinbelow.

In at least some embodiments not shown in FIG. 17, the controller 65 may be configured to control a surgical robotic assembly that may be used to perform the joint arthroplasty robotically.

FIG. 18 is a block diagram of the controller 65 of an improved computer-based platform for classification of total joint arthroplasty patient alignment based on intraoperative data acquisitions in accordance with one or more embodiments of the present disclosure. The controller 65 of the surgery assistance device 12 represented in FIG. 17 may include a processor 70, a memory 80, input and output (I/O) devices 75 such as the display 60 and the keyboard 62, a communication circuitry 90, and a surgical tool and sensor control circuitry 95. The communication circuitry 90 may enable the controller 65 to communicate with other computing devices over any suitable wired and/or wireless communication network. The communication circuitry 90 may be enabled by the controller 65 to communicate with the at least one surgical tool 56A and/or with the at least one surgical probe 56B, and/or the at least one imaging camera 50 and/or with the at least one first tracker 40A and/or the at least one second tracker 40B.

In at least some embodiments, the surgical tool and sensor control circuitry 95 may be configured to process sensor signals from the at least one surgical tool 56A and the at least one surgical probe 56B, and/or the at least one imaging camera 50 and/or with the at least one first tracker 40A and/or the at least one second tracker 40B, and/or for any other suitable surgical devices and/or sensors needed to perform the total joint arthroscopy procedure. In other embodiments, the surgical tool and sensor control circuitry 95 may be configured to receive commands from the processor 70. The commands may be used to control the at least one surgical tool 56A and the at least one surgical probe 56B during surgery, and/or to control a robotic surgical apparatus for performing the surgical total joint arthroscopy procedure in the operating room 10.

In at least some embodiments, the processor 70 may be configured to execute a surgical plan software 72 that may include at least one software module implemented as algorithms, trained machine learning modules, or both. The at least one software module may include algorithms 74 such as a knee phenotype classifier 73 (e.g., a joint phenotype classification algorithm), a joint gap and distraction force simulator 77, a surgical plan generator 78, and/or a GUI manager 79.

In at least some embodiments, the surgical plan generator 78 may be used for generating and/or updating the patient-specific surgical plan in real time so as to assist the surgeon 15 before and/or during the joint arthroplasty procedure. The surgical plan generator 78 may use as inputs any of: joint laxity (joint gap) data under at least one force regime (measured and/or simulated), an implant profile, a surgeon-specific surgery profile, and a patient-specific post-surgery desired functional profile.

In at least some embodiments, the memory 80 may be configured to store a patient data database 81 storing the data from N patients, where N is an integer. The patient data database 81 may include a patient record 82 of patient 1 that includes for patient 1, patient data 83, distraction force and/or joint movement data 84, and a patient-specific surgical plan 85. The patient data database 81 may include a patient record 86 of the Nth patient N that includes for patient N, patient data 87, distraction force and/or joint movement data 88, and a patient-specific surgical plan 89.

In at least some embodiments, the surgical plan generator 78 executed by the processor 70 output a patient-specific surgical plan stored in the patient data database 81. A GUI manager software module 79 may be configured to transmit instructions to the display 60 to display, for example, the patient-specific surgical plan 85 on the GUI 61 for the surgeon 15 to view before and/or during the arthroplasty surgical procedure. All or any of the above software routines may be stored in the memory 80.

In at least some embodiments, bone registration data may be obtained using at least one intra-operative imaging modality such as X-ray imaging, computed tomography (CT) imaging, magnetic resonance imaging (MRI), or any combination thereof.

In at least some embodiments, bone registration data may include geometric points defined along a surface of the bone member which may use medical image data to delineate the bone edge boundaries and other bone features. The bone registration data may be used to model a bone member representation in which the geometric points along the bone edge boundaries and/or other bone features may be defined within a coordinate system. In other embodiments, each bone member representation may be defined in its own unique coordinate system. In yet other embodiments, the first bone member representation may be defined in a single coordinate system.

In at least some embodiments, using the bone registration data, the controller 65 may use a First/Second Bone member representation modeler to generate a first bone member representation of the first bone member and a second bone member representation of the second bone member using the bone registration data.

In at least some embodiments, the surgical plan software 72, as illustrated in FIG. 18, may be stored in the memory 80 and executed by the processor 70 of the controller 65. The surgical plan software 72 may perform functions related to generating the patient-specific intra-operative surgical plan. The software may obtain bone registration data for a first bone member and a second bone member of a joint using input and output (I/O) devices 75 and communication circuitry 90; may acquire, during the surgical procedure, patient-specific movement-related data after the first bone member, the second bone member, or both, have been put through at least one movement where a first predefined distraction force is applied between the first bone member and the second bone member throughout a continuous range of motions, with data received from surgical tools and sensor control circuitry 95.

In at least some embodiments, the surgical plan software 72 may conduct, using a distraction force model, a joint gap simulation (e.g., distraction force/joint gap simulator module 77) based on predefined ligament stiffness data, the patient-specific movement-related data at the first predefined distraction force, and at least one simulated distraction force value; may obtain, from the joint gap simulation, joint gap simulation data; may utilize a surgical plan model (e.g., the surgical plan generator 78) to generate a patient-specific intra-operative surgical plan for the implantation of the implant based at least in part on the joint gap simulation data and the bone registration data for the first bone member and the second bone member, and may control or provide instructions for cutting the first bone member, the second bone member, or both, based at least in part on at least one surgical cut parameter of the patient-specific intra-operative surgical plan to facilitate the implantation of the implant. The generated patient-specific intra-operative surgical plan and related data may be displayed to the surgeon via the display 60 and graphical user interface 61.

In at least some embodiments, the surgical plan software 72 may receive a surgeon-specific surgery profile, a patient-specific post-surgery desired functional profile, implant profiles, bone registration data, patient-specific movement-related data, joint alignment phenotype data from the algorithms 74 including the knee (joint) phenotype classifier 73, and/or joint gap data from the distraction force/joint gap simulator 77 as inputs. The surgical plan generator 78, that includes the surgical plan model, may apply these inputs to the surgical plan model to generate the patient-specific intra-operative surgical plan by evaluating at least a plurality of dependencies between surgical parameters, implant characteristics, expected functional performance of the joint, movement-related data, joint alignment phenotypes, and/or the joint gap data. The model may be designed to achieve the patient-specific post-surgery desired functional profile by determining estimated values for each surgical parameter, such as bone resection depths, alignment angles, and flexion angles, for example, that may be tailored to both the surgeon's preferences and the patient's anatomical and functional requirements.

In at least some embodiments, the surgical plan model may include ranges of acceptable and preferred values for each surgical parameter, which may be defined by the surgeon, the implant manufacturer, or both. The model may process these ranges along with the patient-specific functional objectives, such as targeted joint gaps or alignment, to compute a set of surgical cut parameters that may fall within the defined tolerance bands. The model may also allow for the prioritization or weighting of different functional objectives, so that the surgical plan may reflect the most important clinical outcomes for the patient.

In at least some embodiments, the surgical plan model may be configured to update the patient-specific intra-operative surgical plan in real time as new intra-operative data is acquired. For example, after preliminary bone cuts or soft-tissue assessments, the model may recalculate the optimal surgical parameters based on updated movement-related data or changes in the soft-tissue envelope. The updated plan may be displayed to the surgeon on a graphical user interface, allowing for interactive adjustment of surgical variables and immediate visualization of the predicted impact on joint function.

In at least some embodiments, the surgical plan model may generate the patient-specific intra-operative surgical plan by leveraging machine learning algorithms trained on historical surgical data, patient outcomes, and implant performance. The model may use these data-driven insights to recommend surgical parameters that may optimize both the immediate surgical result and the long-term functional outcome for the patient. The plan may include detailed guidance for bone cuts, implant positioning, and soft-tissue management, and may be presented to the surgeon in a format that highlights key performance indicators such as alignment, balance, and sizing.

In at least some embodiments, the surgical plan model may support the comparison of multiple surgical scenarios by generating alternative patient-specific intra-operative surgical plans based on different input parameters or implant options. The surgeon may review these alternatives on the graphical user interface, compare predicted functional outcomes, and select the most appropriate plan for execution during the procedure.

In at least some embodiments, the surgical plan software 72 may receive, by the controller 65, a patient-specific profile that may include a plurality of patient-specific values for a plurality of patient-specific parameters. These patient-specific parameters may include anatomical measurements, demographic information, preoperative imaging data, and movement-related data unique to the individual patient. The software may use these values as key inputs for generating a surgical plan tailored to the patient's unique anatomy and clinical needs.

In at least some embodiments, the surgical plan software 72 may also receive, by the controller 65, a healthcare-specific profile that may include a plurality of healthcare-specific values for a plurality of healthcare-specific parameters. These healthcare-specific parameters may include surgeon preferences, institutional protocols, implant manufacturer guidelines, and other clinical best practices. The software may use these values to ensure that the generated surgical plan aligns with the standards and requirements of the healthcare provider or institution.

In at least some embodiments, the inputting of the plurality of inputs into the surgical plan model may include inputting both the plurality of patient-specific values for the patient-specific parameters and the plurality of healthcare-specific values for the healthcare-specific parameters into the surgical plan model. The surgical plan model may process these combined inputs to generate the patient-specific intra-operative surgical plan that is both personalized to the patient and consistent with healthcare provider requirements.

In at least some embodiments, the surgical plan model may be designed to achieve the patient-specific post-surgery desired functional profile based at least in part on a plurality of dependencies between any of: the patient-specific parameters, the healthcare-specific parameters, the surgical parameters, at least one functional parameter representative of the expected functional performance of the joint, the movement-related data, the joint alignment phenotype data, and/or the joint gap data. The model may evaluate these dependencies to determine optimal surgical cut parameters, implant selection, and alignment strategies that may maximize the likelihood of achieving the desired clinical and functional outcomes for the patient.

In at least some embodiments, the at least one surgical cut parameter may include a bone resection depth for the first bone member, the second bone member, or both; may include an alignment angle for the bone cuts; may include a flexion angle at which the cuts are performed; may include the thickness of the prosthetic component to be implanted; and may include parameters related to the balancing of soft tissue such as adjustments to achieve targeted joint gaps. The surgical plan model may determine these parameters based on the patient-specific movement-related data, the bone registration data, and the desired functional outcome as well as the joint alignment phenotype data, and/or the joint gap data, so that the resulting patient-specific intra-operative surgical plan may be tailored to the anatomical and clinical needs of the patient.

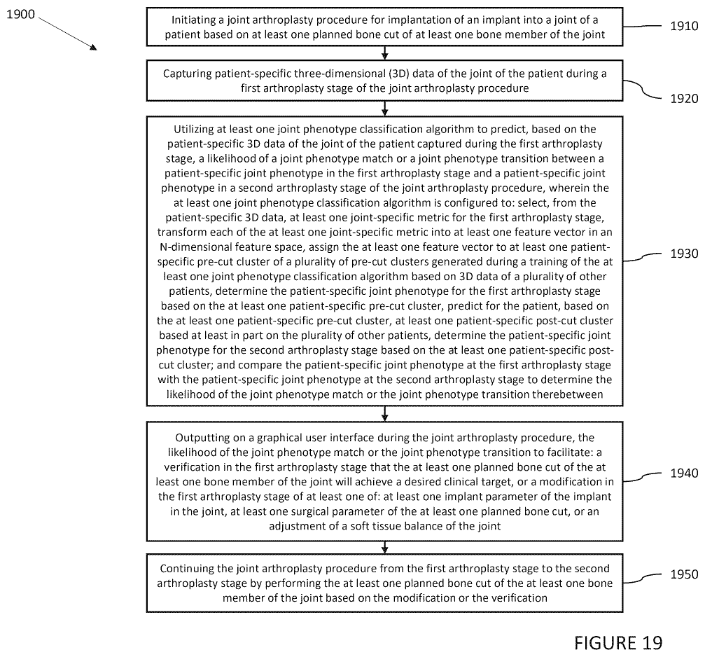

FIG. 19 is a flowchart of a method 1900 for classification of total joint arthroplasty patient alignment based on intraoperative data acquisitions in accordance with one or more embodiments of the present disclosure. In at least some embodiments, the at least one joint phenotype classification algorithm may be performed, for example, by the processor 70 of the controller 65.

In at least some embodiments, the method 1900 may include initiating 1910 a joint arthroplasty procedure for implantation of an implant into a joint of a patient based on at least one planned bone cut of at least one bone member of the joint.

In at least some embodiments, the method 1900 may include capturing 1920 patient-specific three-dimensional (3D) data of the joint of the patient during a first arthroplasty stage of the joint arthroplasty procedure.

In at least some embodiments, the method 1900 may include utilizing 1930 at least one joint phenotype classification algorithm to predict, based on the patient-specific 3D data of the joint of the patient captured during the first arthroplasty stage, a likelihood of a joint phenotype match or a joint phenotype transition between a patient-specific joint phenotype in the first arthroplasty stage and a patient-specific joint phenotype in a second arthroplasty stage of the joint arthroplasty procedure, wherein the at least one joint phenotype classification algorithm is configured to: select, from the patient-specific 3D data, at least one joint-specific metric for the first arthroplasty stage, transform each of the at least one joint-specific metric into at least one feature vector in an N-dimensional feature space, assign the at least one feature vector to at least one patient-specific pre-cut cluster of a plurality of pre-cut clusters generated during a training of the at least one joint phenotype classification algorithm based on 3D data of a plurality of other patients, determine the patient-specific joint phenotype for the first arthroplasty stage based on the at least one patient-specific pre-cut cluster, predict for the patient, based on the at least one patient-specific pre-cut cluster, at least one patient-specific post-cut cluster based at least in part on the plurality of other patients, determine the patient-specific joint phenotype for the second arthroplasty stage based on the at least one patient-specific post-cut cluster; and compare the patient-specific joint phenotype at the first arthroplasty stage with the patient-specific joint phenotype at the second arthroplasty stage to determine the likelihood of the joint phenotype match or the joint phenotype transition therebetween.

In at least some embodiments, the method 1900 may include outputting 1940 on a graphical user interface during the joint arthroplasty procedure, the likelihood of the joint phenotype match or the joint phenotype transition to facilitate: a verification in the first arthroplasty stage that the at least one planned bone cut of the at least one bone member of the joint will achieve a desired clinical target, or a modification in the first arthroplasty stage of at least one of: at least one implant parameter of the implant in the joint, at least one surgical parameter of the at least one planned bone cut, or an adjustment of a soft tissue balance of the joint.

In at least some embodiments, the method 1900 may include continuing 1950 the joint arthroplasty procedure from the first arthroplasty stage to the second arthroplasty stage by performing the at least one planned bone cut of the at least one bone member of the joint based on the modification or the verification.

Note the term “desired clinical target” may refer to a predefined or surgeon-selected patient-specific outcome for the knee joint arthroplasty procedure that may be based on patient-specific anatomical, functional, or biomechanical parameters. The desired clinical target may include, but is not limited to, achieving a specific three-dimensional hip-knee-ankle alignment, restoring native or optimal joint kinematics, attaining balanced soft tissue tension throughout the range of motion, minimizing joint instability, or meeting other objective or subjective criteria that may be associated with improved patient function, satisfaction, and long-term implant survivorship. The desired clinical target may be determined preoperatively, intraoperatively, or postoperatively, and may be tailored to the unique needs, anatomy, and clinical presentation of each patient.

In at least some embodiments, the term “first arthroplasty stage” may include an intraoperative stage that may relate to at least one of: before any cut may be performed on a bone member, or after a cut of a first bone member may be executed but before a cut of a second bone member may be performed. The term “second arthroplasty stage” may include an intraoperative stage that may relate to a state where cuts of two bone members may be completed. The term “pre-cut” may include data acquisitions that may be captured during the first arthroplasty stage and may be used to select at least one feature vector that may be assigned to at least one patient-specific pre-cut cluster that may be generated during training. The term “post-cut” may include data acquisitions that may be captured during the second arthroplasty stage and may be used to determine at least one patient-specific post-cut cluster that may be predicted based at least in part on the pre-cut cluster and historical data. These may be applied to phenotype determination such that a patient-specific joint phenotype at the first arthroplasty stage may be derived from the pre-cut cluster and a patient-specific joint phenotype at the second arthroplasty stage may be derived from the post-cut cluster, which may enable a comparison that may output a likelihood of phenotype match or phenotype transition.

In at least some embodiments, a joint-agnostic phenotype classification pipeline may include selecting at least one joint-specific metric from patient-specific three-dimensional pre-cut data, may include transforming the selected metric into at least one feature vector in an N-dimensional feature space (e.g., a higher-dimensional feature space), where N is an integer, may include assigning the feature vector to at least one patient-specific pre-cut cluster trained on other patients, and may include predicting at least one patient-specific post-cut cluster, where the pipeline may determine a pre-cut and post-cut joint phenotype and may compute a likelihood of a phenotype match or transition for intraoperative decision support.

In at least some embodiments, knee-specific metrics may include coronal varus/valgus alignment, sagittal flexion/extension, and rotational internal/external orientation, where the algorithm may select metrics at predefined knee position states (e.g., extension and flexion) and may generate feature vectors that may capture knee alignment behavior for clustering, prediction, and phenotype comparison.

In at least some embodiments, shoulder-specific metrics may include coronal abduction, sagittal flexion/extension, and rotational internal/external orientation, where the algorithm may select metrics at predefined shoulder position states (e.g., abduction and rotation angles) and may construct feature vectors that may enable clustering of motion or state-conditioned data and may support phenotype prediction and intraoperative guidance.

In at least some embodiments, ankle-specific metrics may include coronal varus/valgus alignment, sagittal dorsiflexion/plantarflexion, and rotational internal/external orientation and inversion/eversion, where the algorithm may select metrics at predefined ankle position states (e.g., dorsiflexion and plantarflexion) and may form feature vectors that may drive clustering, post-cut cluster prediction, and phenotype likelihood computation.

In at least some embodiments, position states may include joint-appropriate predefined angles or ranges (e.g., knee extension and flexion, shoulder abduction and rotation, ankle dorsiflexion and plantarflexion), where the algorithm may define evolution between two position states for dynamic acquisitions and may omit evolution for static state-only acquisitions while still generating phenotypes.

In at least some embodiments, a static implementation may include acquiring joint data at two or more predefined position states without continuous motion, where the algorithm may select the state-conditioned metrics, may include constructing feature vectors from the static states, and may include determining pre-cut clusters, predicting post-cut clusters, and outputting a likelihood of match or transition without generating an evolution class.

In at least some embodiments, model training may include receiving historical pre-cut feature vectors and corresponding pre-cut cluster labels, may include receiving post-cut cluster labels for the same cases, and may include training a model that may map pre-cut feature vectors to a probability distribution over post-cut clusters and a likelihood of phenotype match versus transition, where the trained parameters may be used intraoperatively for patient-specific inference.

In at least some embodiments, intraoperative guidance may include outputting on a graphical user interface a likelihood of phenotype match or transition and may include presenting recommendations to modify at least one implant parameter, at least one surgical cut parameter, or soft-tissue balance, where the GUI may display joint-appropriate position states and metric trends to support verification of a desired clinical target and may enable continuing the procedure according to the verification or modification.

In at least some embodiments, the capturing of patient-specific joint data may include patient-specific three-dimensional alignment data over a full range of motion during a first arthroplasty stage, where the data may include dynamic acquisitions across predefined position states or static acquisitions at selected states to enable joint-agnostic analysis across knee, shoulder, and ankle.

In at least some embodiments, at least one patient-specific pre-cut cluster may include an association with a first joint-specific position state and a second joint-specific position state, where knee states may include extension and flexion, shoulder states may include abduction and rotation, and ankle states may include dorsiflexion and plantarflexion.

In at least some embodiments, the utilization of at least one joint phenotype classification algorithm may include at least one of an unsupervised learning model, a supervised learning model, or a semi-supervised learning model trained on historical joint-specific data from a plurality of other patients, where the training set may include multi-surgeon and multi-implant cases.

In at least some embodiments, at least one joint-specific metric may include at least one joint-specific alignment angle, where the patient-specific three-dimensional data may include patient-specific three-dimensional alignment data, where an N-dimensional motion position feature space may include features at predefined position states, and where the algorithm may select alignment angles at the predefined states and may transform them into at least one feature vector in the N-dimensional motion position feature space.

In at least some embodiments, the joint phenotype classification algorithm may include classifying, based on the at least one patient-specific pre-cut cluster, at least one feature vector into at least two classifications to generate a pre-cut evolution class; may include determining a patient-specific joint phenotype for the first arthroplasty stage based on the pre-cut evolution class; may include predicting, based on the at least one patient-specific pre-cut cluster and the pre-cut evolution class, at least one patient-specific post-cut cluster and a post-cut evolution class; and may include determining a patient-specific joint phenotype for the second arthroplasty stage based on the at least one patient-specific post-cut cluster and the post-cut evolution class.

In at least some embodiments, capturing joint data across the full range of motion may include acquiring joint alignment and load data, where for a lower-limb joint the data may include hip-knee-ankle alignment and associated medial-lateral, anterior-posterior, or axial force or pressure measurements at predetermined flexion or rotation angles during intraoperative manipulation.

In at least some embodiments, the joint phenotype classification algorithm may include applying an unsupervised clustering algorithm to at least one feature vector, where clustering may group similar joint motion or state-conditioned patterns across knee, shoulder, and ankle cohorts.

In at least some embodiments, the joint phenotype classification algorithm may include selecting an unsupervised clustering algorithm from k-means, hierarchical clustering, or Gaussian mixture models, where the selection may be based on data distribution, scalability, and intraoperative latency constraints discussed for deployment.

In at least some embodiments, the joint phenotype classification algorithm may include determining an optimal number of clusters using an Elbow method, a Davies-Bouldin index, a silhouette coefficient, or a Calinski-Harabasz score, where model selection metrics may be computed during offline training on historical cases.

In at least some embodiments, the joint phenotype classification algorithm may include executing a comparison module to facilitate real-time feedback to a surgeon regarding preservation or alteration of a native alignment pattern, where a graphical user interface may display likelihood of match or transition and may prompt verification or modification actions intraoperatively.

In at least some embodiments, the joint phenotype classification algorithm may include analyzing historical case data to identify trends in surgical alignment practices, where retrospective aggregates may reveal surgeon-specific match rates and transition patterns useful for planning and quality improvement.

In at least some embodiments, the joint phenotype classification algorithm may include providing guidance or training to junior surgeons based on phenotype data from expert-led cases, where the system may surface exemplar trajectories and recommended parameter adjustments that may increase phenotype match likelihood for knee, shoulder, and ankle procedures.

In at least some embodiments, a method may include initiating a joint arthroplasty procedure for implantation of an implant into a joint of a patient based on at least one planned bone cut of at least one bone member of the joint; may include capturing patient-specific three-dimensional (3D) data of the joint of the patient during a first arthroplasty stage of the joint arthroplasty procedure; may include utilizing at least one joint phenotype classification algorithm to predict, based on the patient-specific 3D data of the joint of the patient captured during the first arthroplasty stage, a likelihood of a joint phenotype match or a joint phenotype transition between a patient-specific joint phenotype in the first arthroplasty stage and a patient-specific joint phenotype in a second arthroplasty stage of the joint arthroplasty procedure; where the at least one joint phenotype classification algorithm may be configured to: may select, from the patient-specific 3D data, at least one joint-specific metric for the first arthroplasty stage; may transform each of the at least one joint-specific metric into at least one feature vector in an N-dimensional feature space; may assign the at least one feature vector to at least one patient-specific pre-cut cluster of a plurality of pre-cut clusters generated during a training of the at least one joint phenotype classification algorithm based on 3D data of a plurality of other patients; may determine the patient-specific joint phenotype for the first arthroplasty stage based on the at least one patient-specific pre-cut cluster; may predict for the patient, based on the at least one patient-specific pre-cut cluster, at least one patient-specific post-cut cluster based at least in part on the plurality of other patients; may determine the patient-specific joint phenotype for the second arthroplasty stage based on the at least one patient-specific post-cut cluster; and may compare the patient-specific joint phenotype at the first arthroplasty stage with the patient-specific joint phenotype at the second arthroplasty stage to determine the likelihood of the joint phenotype match or the joint phenotype transition therebetween; and may include outputting on a graphical user interface during the joint arthroplasty procedure, the likelihood of the joint phenotype match or the joint phenotype transition to facilitate: may verify in the first arthroplasty stage that the at least one planned bone cut of the at least one bone member of the joint will achieve a desired clinical target, or may modify in the first arthroplasty stage at least one of: at least one implant parameter of the implant in the joint, at least one surgical parameter of the at least one planned bone cut, or an adjustment of a soft tissue balance of the joint; and may include continuing the joint arthroplasty procedure from the first arthroplasty stage to the second arthroplasty stage by performing the at least one planned bone cut of the at least one bone member of the joint based on the modification or the verification.

In at least some embodiments, the capturing of the patient-specific 3D data may include capturing patient-specific 3D alignment data of the joint of the patient over a full range of motion during the first arthroplasty stage of the joint arthroplasty procedure.

In at least some embodiments, the at least one patient-specific pre-cut cluster may be associated with a first joint-specific position state and a second joint-specific position state.

In at least some embodiments, the utilizing of the at least one joint phenotype classification algorithm may include utilizing at least one of: an unsupervised learning model, a supervised learning model, or semi-supervised learning model trained on historical alignment data for the plurality of other patients.

In at least some embodiments, the at least one joint-specific metric may include at least one joint-specific alignment angle; where the patient-specific three-dimensional (3D) data may include patient-specific 3D alignment data; where the N-dimensional feature space may be an N-dimensional motion position feature space; and where the at least one joint phenotype classification algorithm may be configured to: may select, from the patient-specific 3D alignment data, the at least one joint-specific alignment angle over a plurality of predetermined joint-specific motion position angles for the first arthroplasty stage; and may transform each of the at least one joint-specific alignment angle into the at least one feature vector in the N-dimensional motion position feature space.

In at least some embodiments, the at least one joint phenotype classification algorithm may further be configured to: may classify, based on the at least one patient-specific pre-cut cluster, the at least one feature vector into one of at least two alignment classifications to generate a pre-cut evolution class; may determine the patient-specific joint phenotype for the first arthroplasty stage based on the pre-cut evolution class; may predict for the patient, based on the at least one patient-specific pre-cut cluster and the pre-cut evolution class, the at least one patient-specific post-cut cluster and a post-cut evolution class based at least in part on the plurality of other patients; and may determine the patient-specific joint phenotype for the second arthroplasty stage based on the at least one patient-specific post-cut cluster and the post-cut evolution class.

In at least some embodiments, capturing the patient-specific 3D alignment data of the joint over a full range of motion during at least one of the first arthroplasty stage and the second arthroplasty stage may include acquiring joint alignment and load data, including, for a lower-limb joint, hip-knee-ankle alignment data and associated medial-lateral, anterior-posterior, or axial force or pressure measurements at a plurality of predetermined flexion or rotation angles using a computer-assisted orthopedic system during intraoperative manipulation of the joint.

In at least some embodiments, the at least one joint phenotype classification algorithm may be configured to apply an unsupervised clustering algorithm to the at least one feature vector.

In at least some embodiments, the at least one joint phenotype classification algorithm may be configured to select an unsupervised clustering algorithm from the group including k-means, hierarchical clustering, and Gaussian mixture models.

In at least some embodiments, the at least one joint phenotype classification algorithm may be configured to determine an optimal number of clusters using an Elbow method, a Davies-Bouldin index, a silhouette coefficient, or a Calinski-Harabasz score.

In at least some embodiments, the at least one joint phenotype classification algorithm may be configured to execute a comparison module to facilitate real-time feedback to a surgeon regarding preservation or alteration of a native alignment pattern of a patient.

In at least some embodiments, the method may further include utilizing the at least one joint phenotype classification algorithm that may be configured to analyze historical case data to identify trends in surgical alignment practices.

In at least some embodiments, the method may further include utilizing the at least one joint phenotype classification algorithm that may be configured to provide guidance or training to junior surgeons based on phenotype data from expert-led cases.

In at least some embodiments, the joint may be a knee joint.

In at least some embodiments, the at least one joint phenotype classification algorithm may be configured to execute a feature extraction module to interpolate varus-valgus angles from a plurality of predetermined flexion angles.

In at least some embodiments, the at least one joint phenotype classification algorithm may be configured to construct the at least one feature vector for each patient from at least one varus-valgus angle.

In at least some embodiments, the at least one joint phenotype classification algorithm may be configured to classify knee joint classification as one of: varus, neutral, or valgus based on a threshold value of a varus-valgus angle.

In at least some embodiments, the at least one joint phenotype classification algorithm may be configured to generate an evolution class for each patient based on extension and flexion state classifications.

In at least some embodiments, the at least one joint phenotype classification algorithm may be configured to process three-dimensional hip-knee-ankle (HKA) alignment data acquired at a plurality of predetermined flexion angles.

In at least some embodiments, the at least one joint phenotype classification algorithm may be configured to transform a two-dimensional dynamic trajectory of varus-valgus and flexion into a feature vector point in a multi-dimensional feature space.

In at least some embodiments, the at least one joint phenotype classification algorithm may be configured to assign each feature vector to one of the plurality of clusters representing distinct knee motion patterns.

In at least some embodiments, the at least one joint phenotype classification algorithm may be configured to use a phenotype definition module to combine cluster assignment and an evolution class to define the patient-specific joint phenotype.

In at least some embodiments, the at least one joint phenotype classification algorithm may be configured to select varus-valgus angles at flexion angles that may include at least 10°, 15°, 20°, 30°, 45°, 60°, 75°, 90°, and 105°.

In at least some embodiments, the at least one joint phenotype classification algorithm may be configured to generate a graphical output of the patient-specific joint phenotype for display on the graphical user interface.

In at least some embodiments, the capturing of the patient-specific 3D data of the knee joint over a full range of motion at the first arthroplasty stage may include acquiring hip-knee-ankle alignment data at a plurality of predetermined flexion angles using a computer assisted orthopedic system during intraoperative manipulation of the knee joint.

In at least some embodiments, the modification of the at least one implant parameter may include adjusting at least one of: an implant component size, a varus-valgus orientation, a rotational alignment of the implant, or any combination thereof.

In at least some embodiments, the modification of the at least one surgical parameter of the at least one planned bone cut may include modifying the at least one surgical parameter for resecting bone from at least one of: a distal femur, a proximal tibia, or both, to correct a varus or valgus deformity.

In at least some embodiments, the adjustment of the soft tissue balance may include performing at least one of: selectively releasing at least one ligament, tensioning at least one ligament, or any combination thereof, about the knee joint to achieve a target medial-lateral gap.

In at least some embodiments, the verification that the joint arthroplasty procedure achieved the desired clinical target may include acquiring three-dimensional hip-knee-ankle alignment data and verifying that the three-dimensional hip-knee-ankle alignment data corresponds to the desired clinical target.

In at least some embodiments, the joint may be a shoulder joint.

In at least some embodiments, the at least one joint-specific metric may include a glenohumeral abduction/adduction angle, a shoulder flexion/extension angle, an internal/external rotation angle, or any combination thereof, selected at predefined shoulder position states.

In at least some embodiments, capturing the patient-specific three-dimensional data may include acquiring joint alignment, load data, or both, at a plurality of predetermined abduction and rotation angles during intraoperative manipulation of the shoulder joint.

In at least some embodiments, the at least one joint phenotype classification algorithm may be configured to classify shoulder position states between a first abduction state and a second rotation state to generate a shoulder evolution class and to determine a patient-specific shoulder joint phenotype.

In at least some embodiments, the at least one feature vector may include values of shoulder alignment angles, glenohumeral contact point locations, soft-tissue tension surrogates, component rotational alignment, or any combination thereof at predefined abduction and rotation states.

In at least some embodiments, the joint may be an ankle joint.

In at least some embodiments, the at least one joint-specific metric may include an ankle dorsiflexion/plantarflexion angle, an inversion/eversion angle, a varus/valgus alignment value, an internal/external rotation angle, or any combination thereof, selected at predefined ankle position states.

In at least some embodiments, capturing the patient-specific three-dimensional data may include acquiring joint alignment, compartmental pressure, force data, or any combination thereof at a plurality of predetermined dorsiflexion and plantarflexion angles during intraoperative manipulation of the ankle joint.

In at least some embodiments, the at least one joint phenotype classification algorithm may be configured to define ankle position states at dorsiflexion and plantarflexion and to classify evolution between the position states to generate an ankle evolution class and to determine a patient-specific ankle joint phenotype.

In at least some embodiments, the at least one feature vector may include a concatenation of ankle alignment angles, medial and lateral gap magnitudes, and compartmental pressure values at predefined dorsiflexion and plantarflexion states.

In at least some embodiments, the at least one joint phenotype classification algorithm may be configured to predict a probability distribution over post-cut clusters conditioned on at least one of surgeon identity, implant family, or surgical workflow for the joint.

In at least some embodiments, the first arthroplasty stage may be before the at least one planned cut is performed.

In at least some embodiments, the at least one bone member may include a first bone member and a second bone member; where the at least one planned bone cut may include a first bone cut of the first bone member and a second bone cut of the second bone member; and where the first arthroplasty stage may be after the first bone cut was performed and before the second bone cut is performed.

In at least some embodiments, the second arthroplasty stage may be after a first cut of a first bone member and a second cut of a second bone member are performed.

In at least some embodiments, a computer-assisted orthopedic system (CAOS) may include at least one three-dimensional tracking assembly configured to capture patient-specific three-dimensional (3D) data of a joint of a patient during a first arthroplasty stage of a joint arthroplasty procedure for implantation of an implant into the joint of a patient based on at least one planned bone cut of at least one bone member of the joint; where the CAOS may include at least one processor configured to execute instructions stored in at least one non-transitory memory, the instructions may cause the at least one processor to utilize at least one joint phenotype classification algorithm to: may predict, based on the patient-specific 3D data of the joint of the patient captured during the first arthroplasty stage, a likelihood of a joint phenotype match or a joint phenotype transition between a patient-specific joint phenotype in the first arthroplasty stage and a patient-specific joint phenotype in a second arthroplasty stage of the joint arthroplasty procedure; where the at least one joint phenotype classification algorithm may be configured to: may select, from the patient-specific 3D data, at least one joint-specific metric for the first arthroplasty stage; may transform each of the at least one joint-specific metric into at least one feature vector in an N-dimensional feature space; may assign the at least one feature vector to at least one patient-specific pre-cut cluster of a plurality of pre-cut clusters generated during a training of the at least one joint phenotype classification algorithm based on 3D data of a plurality of other patients; may determine the patient-specific joint phenotype for the first arthroplasty stage based on the at least one patient-specific pre-cut cluster; may predict for the patient, based on the at least one patient-specific pre-cut cluster, at least one patient-specific post-cut cluster based at least in part on the plurality of other patients; may determine the patient-specific joint phenotype for the second arthroplasty stage based on the at least one patient-specific post-cut cluster; and may compare the patient-specific joint phenotype at the first arthroplasty stage with the patient-specific joint phenotype at the second arthroplasty stage to determine the likelihood of the joint phenotype match or the joint phenotype transition therebetween; and may include output on a graphical user interface during the joint arthroplasty procedure, the likelihood of the joint phenotype match or the joint phenotype transition to facilitate: may verify in the first arthroplasty stage that the at least one planned bone cut of the at least one bone member of the joint will achieve a desired clinical target, or may modify in the first arthroplasty stage at least one of: at least one implant parameter of the implant in the joint, at least one surgical parameter of the at least one planned bone cut, or an adjustment of a soft tissue balance of the joint; and may include to facilitate a continuation of the joint arthroplasty procedure from the first arthroplasty stage to the second arthroplasty stage by performing the at least one planned bone cut of the at least one bone member of the joint based on the modification or the verification.

In at least some embodiments, the three-dimensional tracking assembly may include any combination of hardware and software components that may be configured to capture patient-specific three-dimensional alignment data of a knee joint over a full range of motion during a first arthroplasty stage and a second arthroplasty stage of a knee joint arthroplasty procedure. The three-dimensional tracking assembly may include at least one imaging camera that may be configured to detect and record the spatial position and orientation of objects within the surgical field. The tracking assembly may further include at least one tracking device that may be attached or rigidly mounted to one or more bone members, such as the femur and tibia, and may be configured to provide real-time positional data to the imaging camera.

In at least some embodiments, the three-dimensional tracking assembly may include additional sensors, markers, or reference arrays that may enhance the accuracy and reliability of the captured data. The tracking assembly may be operatively connected to a controller or processing unit that may process the acquired signals and may generate three-dimensional alignment information for use during the surgical procedure.

In at least some embodiments, the three-dimensional tracking assembly may be integrated with the CAOS as described above and may support intraoperative visualization, navigation, and real-time feedback to a medical practitioner.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the inventive systems/platforms, and the inventive devices described herein can be utilized in any combination with each other. Further still, the various steps may be carried out in any desired order (and any desired steps may be added and/or any desired steps may be eliminated).

1. A method, comprising:
initiating a joint arthroplasty procedure for implantation of an implant into a joint of a patient based on at least one planned bone cut of at least one bone member of the joint;
capturing patient-specific three-dimensional (3D) data of the joint of the patient during a first arthroplasty stage of the joint arthroplasty procedure;
utilizing at least one joint phenotype classification algorithm to predict, based on the patient-specific 3D data of the joint of the patient captured during the first arthroplasty stage, a likelihood of a joint phenotype match or a joint phenotype transition between a patient-specific joint phenotype in the first arthroplasty stage and a patient-specific joint phenotype in a second arthroplasty stage of the joint arthroplasty procedure;
wherein the at least one joint phenotype classification algorithm is configured to:
select, from the patient-specific 3D data, at least one joint-specific metric for the first arthroplasty stage;
transform each of the at least one joint-specific metric into at least one feature vector in an N-dimensional feature space;
assign the at least one feature vector to at least one patient-specific pre-cut cluster of a plurality of pre-cut clusters generated during a training of the at least one joint phenotype classification algorithm based on 3D data of a plurality of other patients;
determine the patient-specific joint phenotype for the first arthroplasty stage based on the at least one patient-specific pre-cut cluster;
predict for the patient, based on the at least one patient-specific pre-cut cluster, at least one patient-specific post-cut cluster based at least in part on the plurality of other patients;
determine the patient-specific joint phenotype for the second arthroplasty stage based on the at least one patient-specific post-cut cluster; and
compare the patient-specific joint phenotype at the first arthroplasty stage with the patient-specific joint phenotype at the second arthroplasty stage to determine the likelihood of the joint phenotype match or the joint phenotype transition therebetween;
outputting on a graphical user interface during the joint arthroplasty procedure, the likelihood of the joint phenotype match or the joint phenotype transition to facilitate:
a verification in the first arthroplasty stage that the at least one planned bone cut of the at least one bone member of the joint will achieve a desired clinical target, or
a modification in the first arthroplasty stage of at least one of:
at least one implant parameter of the implant in the joint,
at least one surgical parameter of the at least one planned bone cut, or
an adjustment of a soft tissue balance of the joint; and
continuing the joint arthroplasty procedure from the first arthroplasty stage to the second arthroplasty stage by performing the at least one planned bone cut of the at least one bone member of the joint based on the modification or the verification.

2. The method according to claim 1, wherein the capturing of the patient-specific 3D data comprises capturing patient-specific 3D alignment data of the joint of the patient over a full range of motion during the first arthroplasty stage of the joint arthroplasty procedure.

3. The method according to claim 1, wherein the at least one patient-specific pre-cut cluster is associated with a first joint-specific position state and a second joint-specific position state.

4. The method of claim 1, wherein the utilizing of the at least one joint phenotype classification algorithm comprises utilizing at least one of: an unsupervised learning model, a supervised learning model, or semi-supervised learning model trained on historical alignment data for the plurality of other patients.

5. The method according to claim 1, wherein the at least one joint-specific metric comprises at least one joint-specific alignment angle;
wherein the patient-specific three-dimensional (3D) data comprises patient-specific 3D alignment data;
wherein the N-dimensional feature space is an N-dimensional motion position feature space; and
wherein the at least one joint phenotype classification algorithm is configured to:
select, from the patient-specific 3D alignment data, the at least one joint-specific alignment angle over a plurality of predetermined joint-specific motion position angles for the first arthroplasty stage; and
transform each of the at least one joint-specific alignment angle into the at least one feature vector in the N-dimensional motion position feature space.

6. The method of claim 5, wherein the at least one joint phenotype classification algorithm is further configured to:
classify, based on the at least one patient-specific pre-cut cluster, the at least one feature vector into one of at least two alignment classifications to generate a pre-cut evolution class;
determine the patient-specific joint phenotype for the first arthroplasty stage based on the pre-cut evolution class;
predict for the patient, based on the at least one patient-specific pre-cut cluster and the pre-cut evolution class, the at least one patient-specific post-cut cluster and a post-cut evolution class based at least in part on the plurality of other patients; and
determine the patient-specific joint phenotype for the second arthroplasty stage based on the at least one patient-specific post-cut cluster and the post-cut evolution class.

7. The method of claim 5, wherein capturing the patient-specific 3D alignment data of the joint over a full range of motion during at least one of the first arthroplasty stage and the second arthroplasty stage comprises acquiring joint alignment and load data, including, for a lower-limb joint, hip-knee-ankle alignment data and associated medial-lateral, anterior-posterior, or axial force or pressure measurements at a plurality of predetermined flexion or rotation angles using a computer-assisted orthopedic system during intraoperative manipulation of the joint.

8. The method of claim 1, wherein the at least one joint phenotype classification algorithm is configured to apply an unsupervised clustering algorithm to the at least one feature vector.

9. The method of claim 1, wherein the at least one joint phenotype classification algorithm is configured to select an unsupervised clustering algorithm from the group consisting of k-means, hierarchical clustering, and Gaussian mixture models.

10. The method of claim 1, wherein the at least one joint phenotype classification algorithm is configured to determine an optimal number of clusters using an Elbow method, a Davies-Bouldin index, a silhouette coefficient, or a Calinski-Harabasz score.

11. The method of claim 1, wherein the at least one joint phenotype classification algorithm is configured to execute a comparison module to facilitate real-time feedback to a surgeon regarding preservation or alteration of a native alignment pattern of a patient.

12. The method of claim 1, further comprising utilizing the at least one joint phenotype classification algorithm that is configured to analyze historical case data to identify trends in surgical alignment practices.

13. The method of claim 1, further comprising utilizing the at least one joint phenotype classification algorithm that is configured to provide guidance or training to junior surgeons based on phenotype data from expert-led cases.

14. The method of claim 1, wherein the joint is a knee joint.

15. The method of claim 14, wherein the at least one joint phenotype classification algorithm is configured to execute a feature extraction module to interpolate varus-valgus angles from a plurality of predetermined flexion angles.

16. The method of claim 14, wherein the at least one joint phenotype classification algorithm is configured to construct the at least one feature vector for each patient from at least one varus-valgus angle.

17. The method of claim 14, wherein the at least one joint phenotype classification algorithm is configured to classify knee joint classification as one of: varus, neutral, or valgus based on a threshold value of a varus-valgus angle.

18. The method of claim 14, wherein the at least one joint phenotype classification algorithm is configured to generate an evolution class for each patient based on extension and flexion state classifications.

19. The method of claim 14, wherein the at least one joint phenotype classification algorithm is configured to process three-dimensional hip-knee-ankle (HKA) alignment data acquired at a plurality of predetermined flexion angles.

20. The method of claim 14, wherein the at least one joint phenotype classification algorithm is configured to transform a two-dimensional dynamic trajectory of varus-valgus and flexion into a feature vector point in a multi-dimensional feature space.

21. The method of claim 14, wherein the at least one joint phenotype classification algorithm is configured to assign each feature vector to one of the plurality of clusters representing distinct knee motion patterns.

22. The method of claim 14, wherein the at least one joint phenotype classification algorithm is configured to use a phenotype definition module to combine cluster assignment and an evolution class to define the patient-specific joint phenotype.

23. The method of claim 14, wherein the at least one joint phenotype classification algorithm is configured to select varus-valgus angles at flexion angles that includes at least 10°, 15°, 20°, 30°, 45°, 60°, 75°, 90°, and 105°.

24. The method of claim 14, wherein the at least one joint phenotype classification algorithm configured to generate a graphical output of the patient-specific joint phenotype for display on the graphical user interface.

25. The method of claim 14, wherein the capturing of the patient-specific 3D data of the knee joint over a full range of motion at the first arthroplasty stage comprises acquiring hip-knee-ankle alignment data at a plurality of predetermined flexion angles using a computer assisted orthopedic system during intraoperative manipulation of the knee joint.

26. The method of claim 14, wherein the modification of the at least one implant parameter comprises adjusting at least one of: an implant component size, a varus-valgus orientation, a rotational alignment of the implant, or any combination thereof.

27. The method of claim 14, wherein the modification of the at least one surgical parameter of the at least one planned bone cut comprises modifying the at least one surgical parameter for resecting bone from at least one of: a distal femur, a proximal tibia, or both, to correct a varus or valgus deformity.

28. The method of claim 14, wherein the adjustment of the soft tissue balance comprises performing at least one of: selectively releasing at least one ligament, tensioning at least one ligament, or any combination thereof, about the knee joint to achieve a target medial-lateral gap.

29. The method according to claim 14, wherein the verification that the joint arthroplasty procedure achieved the desired clinical target comprises acquiring three-dimensional hip-knee-ankle alignment data and verifying that the three-dimensional hip-knee-ankle alignment data corresponds to the desired clinical target.

30. The method of claim 1, wherein the joint is a shoulder joint.

31. The method of claim 30, wherein the at least one joint-specific metric comprises a glenohumeral abduction/adduction angle, a shoulder flexion/extension angle, an internal/external rotation angle, or any combination thereof, selected at predefined shoulder position states.

32. The method of claim 30, wherein capturing the patient-specific three-dimensional data comprises acquiring joint alignment, load data, or both, at a plurality of predetermined abduction and rotation angles during intraoperative manipulation of the shoulder joint.

33. The method of claim 30, wherein the at least one joint phenotype classification algorithm is configured to classify shoulder position states between a first abduction state and a second rotation state to generate a shoulder evolution class and to determine a patient-specific shoulder joint phenotype.

34. The method of claim 30, wherein the at least one feature vector comprises values of shoulder alignment angles, glenohumeral contact point locations, soft-tissue tension surrogates, component rotational alignment, or any combination thereof at predefined abduction and rotation states.

35. The method of claim 1, wherein the joint is an ankle joint.

36. The method of claim 35, wherein the at least one joint-specific metric comprises an ankle dorsiflexion/plantarflexion angle, an inversion/eversion angle, a varus/valgus alignment value, an internal/external rotation angle, or any combination thereof, selected at predefined ankle position states.

37. The method of claim 35, wherein capturing the patient-specific three-dimensional data comprises acquiring joint alignment, compartmental pressure, force data, or any combination thereof at a plurality of predetermined dorsiflexion and plantarflexion angles during intraoperative manipulation of the ankle joint.

38. The method of claim 35, wherein the at least one joint phenotype classification algorithm is configured to define ankle position states at dorsiflexion and plantarflexion and to classify evolution between the position states to generate an ankle evolution class and to determine a patient-specific ankle joint phenotype.

39. The method of claim 35, wherein the at least one feature vector comprises a concatenation of ankle alignment angles, medial and lateral gap magnitudes, and compartmental pressure values at predefined dorsiflexion and plantarflexion states.

40. The method of claim 1, wherein the at least one joint phenotype classification algorithm is configured to predict a probability distribution over post-cut clusters conditioned on at least one of surgeon identity, implant family, or surgical workflow for the joint.

41. The method of claim 1, wherein the first arthroplasty stage is before the at least one planned cut is performed.

42. The method of claim 1, wherein the at least one bone member comprises a first bone member and a second bone member;
wherein the at least one planned bone cut comprises a first bone cut of the first bone member and a second bone cut of the second bone member; and
wherein the first arthroplasty stage is after the first bone cut was performed and before the second bone cut is performed.

43. The method of claim 1, wherein the second arthroplasty stage is after a first cut of a first bone member and a second cut of a second bone member are performed.

44. A computer-assisted orthopedic system (CAOS), comprising:
at least one three-dimensional tracking assembly configured to capture patient-specific three-dimensional (3D) data of a joint of a patient during a first arthroplasty stage of a joint arthroplasty procedure for implantation of an implant into the joint of a patient based on at least one planned bone cut of at least one bone member of the joint;
wherein the CAOS comprises at least one processor configured to execute instructions stored in at least one non-transitory memory, the instructions causing the at least one processor to utilize at least one joint phenotype classification algorithm to:
predict, based on the patient-specific 3D data of the joint of the patient captured during the first arthroplasty stage, a likelihood of a joint phenotype match or a joint phenotype transition between a patient-specific joint phenotype in the first arthroplasty stage and a patient-specific joint phenotype in a second arthroplasty stage of the joint arthroplasty procedure;
wherein the at least one joint phenotype classification algorithm is configured to:
select, from the patient-specific 3D data, at least one joint-specific metric for the first arthroplasty stage;
transform each of the at least one joint-specific metric into at least one feature vector in an N-dimensional feature space;
assign the at least one feature vector to at least one patient-specific pre-cut cluster of a plurality of pre-cut clusters generated during a training of the at least one joint phenotype classification algorithm based on 3D data of a plurality of other patients;
determine the patient-specific joint phenotype for the first arthroplasty stage based on the at least one patient-specific pre-cut cluster;
predict for the patient, based on the at least one patient-specific pre-cut cluster, at least one patient-specific post-cut cluster based at least in part on the plurality of other patients;
determine the patient-specific joint phenotype for the second arthroplasty stage based on the at least one patient-specific post-cut cluster; and
compare the patient-specific joint phenotype at the first arthroplasty stage with the patient-specific joint phenotype at the second arthroplasty stage to determine the likelihood of the joint phenotype match or the joint phenotype transition therebetween; and
output on a graphical user interface during the joint arthroplasty procedure, the likelihood of the joint phenotype match or the joint phenotype transition to facilitate:
a verification in the first arthroplasty stage that the at least one planned bone cut of the at least one bone member of the joint will achieve a desired clinical target, or
a modification in the first arthroplasty stage of at least one of:
at least one implant parameter of the implant in the joint,
at least one surgical parameter of the at least one planned bone cut, or
an adjustment of a soft tissue balance of the joint; and
to facilitate a continuation of the joint arthroplasty procedure from the first arthroplasty stage to the second arthroplasty stage by performing the at least one planned bone cut of the at least one bone member of the joint based on the modification or the verification.
